(19) United States
(12) Patent Application Publication
(10) Pub. No.: US 2023/0317230 A1
(43) Pub. Date: Oct. 5, 2023

(54) METHOD FOR TREATING OBESITY

(71) Applicant: Closed Loop Medicine Ltd., Cambridge (GB)

(72) Inventors: Felicity Kate SARTAIN, London (GB); David COX, London (GB); Paul GOLDSMITH, London (GB); Hakim Adam YADI, London (GB); Andrew John McGlashan RICHARDS, Cambridge (GB); David O'REGAN, London (GB); Bruce CAMPBELL, Cambridge (GB); Michael CATT, Cambridge (GB)

(21) Appl. No.: 18/205,667

(22) Filed: Jun. 5, 2023

Related U.S. Application Data
(63) Continuation-in-part of application No. 16/860,665, filed on Apr. 28, 2020, now Pat. No. 11,710,547.
Application No. 16/860,665 is a continuation-in-part of application No. 17/851,164, filed on Jun. 28, 2022.
Application No. 17/851,164 is a continuation of application No. 16/860,665, filed on Apr. 28, 2020, now Pat. No. 11,710,547.
(60) Provisional application No. 62/841,967, filed on May 2, 2019.
(60) Provisional application No. 62/841,967, filed on May 2, 2019.

(51) Int. Cl. G16H 20/10; G16H 40/67; G16H 50/50; G16H 10/60; G16H 50/20; G16H 20/70; A61B 5/00; A61B 5/021; A61B 5/024; A61B 5/145; A61K 31/4045; A61K 31/4418; A61K 31/485

(52) U.S. Cl. CPC G16H 20/10; G16H 40/67; G16H 50/50; G16H 10/60; G16H 50/20; G16H 20/70; A61B 5/0022; A61B 5/021; A61B 5/024; A61B 5/14532; A61B 5/4824; A61B 5/4848; A61B 5/7264; A61K 31/4045; A61K 31/4418; A61K 31/485; A61B 2560/0252

(57) ABSTRACT

A method for treating obesity in a patient, the method comprising administering a dose of a GLP1 agonist to the patient in a treatment regimen effective to manage the patient's satiety and thereby effect a reduction in the patient's weight; and, in conjunction with the administration of the dose of GLP1 agonist, engaging in a directed digital therapeutic program that manages patient weight loss and the treatment regimen for the GLP1 agonist with the objective of achieving a predetermined target weight for the patient, wherein the patient achieves the target weight.

### CROSS-REFERENCE TO RELATED APPLICATIONS

The present application is a continuation in part of U.S. application Ser. No. 16/860,665, filed Apr. 28, 2020, and entitled “Methods and Systems for Providing Personalised Medicine to a Patient”, and U.S. application Ser. No. 17/851,164, filed Jun. 28, 2022, and entitled “Methods and Systems for Providing Personalised Medicine to a Patient”. Both of these applications in turn claim priority from U.S. Provisional Application No. 62/841,967, filed May 2, 2019, and entitled “Methods and Systems for Providing Personalised Medicine to a Patient”. The present application incorporates all of these referenced applications by reference in their entirety.

### BACKGROUND OF THE INVENTION

The present invention relates to methods and systems suitable for use in identifying and providing personalised medicine to a patient in need thereof, particularly in which a co-therapy is to be used wherein two or more two dosage regimens are provided. The methods and systems may be used to assess the efficacy of dosage forms in a patient utilising a variety of data inputs to provide the personalised medicine. It may also predict suitable dosage regimens for a particular patient.

Patients are routinely prescribed medicine by healthcare providers for the treatment of a range of diseases and conditions. The medicine is to be taken by the patient in accordance with instructions provided by a healthcare provider, which together form a dosage regimen. The dosage regimen is based upon clinical trials that are conducted on a group of patients, and in which the effect of one medicine is compared to another. Clinical trials provide dosage regimens that are generic and not personalised to the particular patient requiring treatment.

Further, patients do not typically have immediate access to healthcare providers. This means that they often have to wait weeks for a new appointment before they are able to discuss their treatment with their healthcare provider, and modify their treatment regimen.

To exacerbate this situation, combinations of therapies (co-therapy regimens) are far less studied than monotherapies, with healthcare providers often prescribing therapies comprising combinations of medicines that have not necessarily been through rigorous clinical trials. Further, NICE guidelines for medicines are usually specific to a particular condition, whereas patients usually suffer co-morbidities.

This means that patients prescribed co-therapies may experience lower levels of care than could be offered by treatment regimens tailored specifically for the individual patient. This may increase health complications and delay or prevent successful treatment of the disease or condition. It may also lead to a decrease in patient compliance as the patient does not feel that the treatment is working or is suitable for them.

In addition to the above, there are other reasons for non-adherence of patients to their medication. These include patients forgetting to take their medicines, off-putting side effects, a lack of tangible efficacy of the medication, greater than once daily frequency of administration, inability to understand complex dosing instructions, and patients exercising their prerogative of choice for a variety of personal or social reasons.

Furthermore, over time the efficacy of a particular medication, or the patient's perception of the efficacy, may decrease due to changes in the patient that are caused by factors unrelated to the medication itself. For example, changes in the lifestyle of the patient may affect their perception of efficacy of the medication, or the actual efficacy of the medication. This may discourage the patient from continuing with the course of treatment. Currently there is no way to capture the complex interdependency between the overall state of the patient and the efficacy of their medication, nor is there a way to determine or predict how a change to the state of the patient may impinge upon the actual or perceived efficacy of the medication.

In view of the above, there is a need in the art to provide methods and systems for providing personalised medicine and address one or more of the above-mentioned problems. There is also the need to provide methods and systems for monitoring and providing co-therapies for “at risk” individuals, such as those with co-morbidities, in drug rehabilitation, with psychological vulnerabilities or with compromised immune systems.

The natural GLP1 (Glucagon-like peptide-1) hormone suppresses appetite and increases satiety. GLP1 slows gastric emptying and may additionally contribute to satiation via increased thermogenesis.

GLP1 agonists (Glucagon-like peptide-1 receptor agonists) are a synthetic analogue derivative of the natural GLP1 hormone which have been modified to increase stability in the human body. GLP1 agonists may be provided with drug label instructions having a fixed dosing regimen which advises use in combination with multidisciplinary dietary advice, including from a dietitian. These nondrug therapies are delivered either face to face or with a diet app, which is a standalone treatment independent of the drug dosage regimen.

### SUMMARY OF THE INVENTION

The present invention provides a reliable and efficient means for providing personalised medicine to a patient in need thereof.

According to a first aspect of the invention, there is provided a method of generating a co-therapy regimen for a patient suffering from a disease or condition, the method comprising the steps of:
a) establishing a desired patient endpoint;
b) identifying the patient position relative to the desired patient endpoint;
c) generating or modifying a dataset relating to the patient, based on one or more patient-related measurements; and
d) processing the dataset, the patient position and the desired patient endpoint to generate the co-therapy regimen.

The method of the first aspect of the invention provides a personalised co-therapy regimen by accurately predicting the co-therapy regimen that is suitable to treat a disease in a particular patient based upon data related to that patient.

According to a second aspect of the invention, there is provided a method of treating a patient suffering from a disease or condition, the method comprising the steps of:
a) selecting a co-therapy suitable to treat the disease or condition;
b) establishing a desired patient endpoint;
c) identifying the patient position relative to the desired patient endpoint;
d) generating or modifying a dataset relating to the patient, based on one or more patient-related measurements;
e) processing the dataset, the patient position and the desired patient endpoint to produce a regimen for the co-therapy; and
f) administering the co-therapy to the patient according to the regimen.

According to one or more embodiments of the second aspect: the disease or condition is obesity and the co-therapy comprises a GLP1-agonist and cognitive behavioural therapy;
the disease or condition is obesity and the co-therapy comprises a GLP1-agonist and metformin; or
the disease or condition is obesity and the co-therapy comprises a GLP1-agonist, metformin and cognitive behavioural therapy.

The second aspect of the invention provides a personalised method of treating a patient suffering from a disease or condition. This may be in the form of an iterative process in which a co-therapy is administered to a patient, and then additional data related to the patient is processed to provide a modified co-therapy. This helps to maintain the optimal treatment of the disease or condition in the dynamic patient environment.

According to a third aspect of the invention, there is provided a system for generating a co-therapy regimen for a patient suffering from a disease or condition, the system comprising at least one data processing device having at least one processor, wherein the system is configured to:
receive an identification of a co-therapy suitable to treat the disease or condition;
receive a desired patient endpoint and a patient position, wherein the patient position is defined relative to the desired patient endpoint;
store a dataset relating to the patient, the dataset comprising one or more patient data based on patient-related measurements;
process the dataset, the patient position and the desired patient endpoint to generate a regimen for the co-therapy; and
output the regimen.

The system of the third aspect of the invention provides a system capable of capturing data relating to the state of the patient and analysing this data to characterise the state of the patient. Based on this characterisation, a regimen for the co-therapy can be generated and outputted by the system, where this regimen is personalised to the current state of the patient. The system can iteratively re-assess the patient state and update the co-therapy regimen where necessary. This helps to maintain the optimal treatment of the disease or condition in the dynamic patient environment.

According to a fourth aspect of the present invention there is provided a method for treating obesity in a patient, the method comprising:
administering a dose of a GLP1 agonist to the patient in a treatment regimen; and,
in conjunction with the administration of the dose of GLP1 agonist, engaging in a directed digital therapeutic program that manages patient weight loss and the treatment regimen for the GLP1 agonist with the objective of achieving a predetermined target weight for the patient.

The method of the fourth aspect advantageously coordinates the drug therapy and the digital therapy to achieve a target weight loss. The method can advantageously synchronise the timing of a behavioural regimen provided by an electronic device with the GLP1 treatment regimen, resulting in strong patient compliance with the treatment program and effective weight loss.

The digital therapeutic program may comprise a behavioural regimen for managing the patient weight loss.

The digital therapeutic program may synchronise a timing of the behavioural regimen with a timing of the administration of the dose of the GLP1 agonist.

The behavioural regimen may comprise a calorie intake regimen and/or a physical activity regimen.

The digital therapeutic program may manage the patient weight loss and the treatment regimen by:
receiving a patient initial weight; and
setting a calorie intake regimen based on the patient initial weight and the target weight.

The digital therapeutic program may manage the patient weight loss and the treatment regimen by:
setting a calorie intake allowance of the calorie intake regimen based on a GLP1 dosage of the treatment regimen.

The method may set a timing of the calorie intake regimen in accordance with a timing of a satiety effect of the GLP1 dosage.

The digital therapeutic program may manage the patient weight loss and the treatment regimen by:
receiving patient progress data comprising one or more of: patient weight loss data, patient calorie intake data, patient motivation score, patient satiety score, patient side effect data and patient activity data;
adjusting a GLP1 dosage of the treatment regimen and/or the calorie intake regimen based on the patient progress data.

The method may comprise adjusting the GLP1 dosage if an elapsed time exceeds a dosage effect time threshold.

The digital therapeutic program may manage the patient weight loss and the treatment regimen by:
during a weight loss phase of the treatment, adjusting a GLP1 dosage of the treatment regimen and/or the calorie intake regimen based on the patient progress data to provide a weight loss trajectory towards the target weight.

The digital therapeutic program may manage the patient weight loss and the treatment regimen by transitioning from a weight loss phase to a weight maintenance phase if the patient weight loss data indicates a rate of weight loss has been less than a threshold weight loss rate for a period of time exceeding a stability time threshold.

The digital therapeutic program may manage the patient weight loss and the treatment regimen by:
during a weight maintenance phase of the treatment, adjusting a GLP1 dosage of the treatment regimen and/or the calorie intake regimen based on the patient progress data to maintain the patient weight within a threshold range of the target weight.

The digital therapeutic program may manage the patient weight loss and the treatment regimen by:
during a weight loss phase of the treatment or a weight maintenance phase of the treatment, adjusting a GLP1 dosage of the treatment regimen to maintain the patient satiety within a threshold patient satiety range.

The digital therapeutic program may manage the patient weight loss and the treatment regimen by:
during a drug withdrawal phase of the treatment, reducing a GLP1 dosage of the treatment regimen and adjusting the calorie intake regimen based on the patient progress data to maintain the patient weight within a threshold range of the target weight and stop administration of the GLP1 agonist.

The digital therapeutic program may manage the patient weight loss and the treatment regimen by:
increasing a calorie intake allowance of the calorie intake regimen if:
   the patient calorie intake data represents a calorie intake greater than a first upper calorie intake threshold;
   the patient motivation score is less than a motivation score threshold; or
   the patient satiety/hunger score is less than a first lower patient satiety threshold.

The digital therapeutic program may manage the patient weight loss and the treatment regimen by temporarily increasing the calorie intake allowance of the calorie intake regimen until an increase in a GLP1 dosage of the treatment regimen takes effect.

The digital therapeutic program may manage the patient weight loss and the treatment regimen by:

increasing a calorie intake allowance of the calorie intake regimen and increasing a GLP1 dosage of the treatment regimen if:
the patient calorie intake data represents a calorie intake greater than a second upper calorie intake threshold;
the patient motivation score is less than a motivation score threshold; or
the patient satiety score is less than a second lower patient satiety threshold.

The digital therapeutic program may manage the patient weight loss and the treatment regimen by:
increasing a GLP1 dosage of the treatment regimen if:

the patient weight loss data represents a weight loss that is less than an acceptable weight loss trajectory;

the patient calorie intake data represents a calorie intake greater than a third upper calorie intake threshold; or

the patient satiety score is less than a third lower patient satiety threshold.

The digital therapeutic program may manage the patient weight loss and the treatment regimen by:
decreasing a GLP1 dosage of the treatment regimen if:

the patient weight loss data represents a weight loss greater than an acceptable weight loss trajectory; or

the patient side effect data is representative of a level of side effects greater than a side effect intolerance threshold.

The digital therapeutic program may manage the patient weight loss and the treatment regimen by:
decreasing a calorie intake allowance of the calorie intake regimen if: the patient calorie intake data represents a calorie intake less than a first lower calorie intake threshold;
the patient motivation score is greater than a motivation score threshold; or
the patient satiety score is greater than a first upper patient satiety threshold.

The digital therapeutic program may manage the patient weight loss and the treatment regimen by:
receiving patient weight loss data; and
setting a patient physical activity regimen based on the patient weight loss data.

The method may comprise setting the physical activity regimen if the weight loss data indicates a weight loss satisfying a weight loss milestone.

The method may comprise setting an intensity of the physical activity regimen based on the patient weight loss data.

According to a fifth aspect of the present invention there is provided a method for providing a co-therapy to a patient suffering from obesity, wherein the co-therapy comprises a GLP1 agonist for administering to the patient according to a treatment regimen and a digital therapeutic program comprising a behavioural regimen for administering using an electronic device, wherein the method comprises:
receiving personalised patient data;
setting a GLP1 dosage of the treatment regimen based on the personalised patient data;
setting a calorie intake regimen of the behavioural regimen based on the personalised patient data;
outputting the GLP1 dosage and the calorie intake regimen.

The method of the fifth aspect can advantageously provide a personalised co-therapy for patients. The method can advantageously synchronise the timing of a behavioural regimen provided by an electronic device with the GLP1 treatment regimen, resulting in strong patient compliance with the treatment program and effective weight loss.

The method may comprise synchronises a timing of the behavioural regimen with a timing of the administration of the dose of the GLP1 agonist.

The method may comprise setting a timing of the calorie intake regimen in accordance with a timing of a satiety effect of the GLP1 dosage.

The personalised patient data may comprise an initial weight and a target weight for the patient.

The personalised patient data may comprise patient progress data comprising one or more of: patient weight loss data, patient calorie intake data, patient motivation score, patient satiety score; patient side effect data; and patient activity data.

The method may comprise adjusting the GLP1 dosage based on the patient progress data if an elapsed time exceeds a dosage effect time threshold.

The digital therapeutic program may manage the patient weight loss and the treatment regimen by:
during a weight loss phase of the treatment, adjusting a GLP1 dosage of the treatment regimen and/or the calorie intake regimen based on the patient progress data to provide a weight loss trajectory towards the target weight.

The digital therapeutic program may manage the patient weight loss and the treatment regimen by transitioning from a weight loss phase to a weight maintenance phase if the patient weight loss data indicates a rate of weight loss has been less than a threshold weight loss rate for a period of time exceeding a stability time threshold.

The digital therapeutic program may manage the patient weight loss and the treatment regimen by:

during a weight maintenance phase of the treatment, adjusting a GLP1 dosage of the treatment regimen and/or the calorie intake regimen based on the patient progress data to maintain the patient weight within a threshold range of the target weight.

The digital therapeutic program may manage the patient weight loss and the treatment regimen by:

during a weight loss phase of the treatment or a weight maintenance phase of the treatment, adjusting a GLP1 dosage of the treatment regimen to maintain the patient satiety within a threshold patient satiety range.

The digital therapeutic program may manage the patient weight loss and the treatment regimen by:

during a drug withdrawal phase of the treatment, reducing a GLP1 dosage of the treatment regimen and adjusting the calorie intake regimen based on the patient progress data to maintain the patient weight within a threshold range of the target weight and stop administration of the GLP1 agonist.

The digital therapeutic program may manage the patient weight loss and the treatment regimen by:
increasing a calorie intake allowance of the calorie intake regimen if:
   the patient calorie intake data represents a calorie intake greater than a first upper calorie intake threshold;
   the patient motivation score is less than a motivation score threshold; or
   the patient satiety score is less than a first lower patient satiety threshold.

The digital therapeutic program may manage the patient weight loss and the treatment regimen by temporarily increasing the calorie intake allowance of the calorie intake regimen until an increase in a GLP1 dosage of the treatment regimen takes effect.

The digital therapeutic program may manage the patient weight loss and the treatment regimen by:

increasing a calorie intake allowance of the calorie intake regimen and increasing a GLP1 dosage of the treatment regimen if:
the patient calorie intake data represents a calorie intake greater than a second upper calorie intake threshold;
the patient motivation score is less than a motivation score threshold; or
the patient satiety score is less than a second lower patient satiety threshold.

The digital therapeutic program may manage the patient weight loss and the treatment regimen by:
increasing a GLP1 dosage of the treatment regimen if:

the patient weight loss data represents a weight loss less than an acceptable weight loss trajectory;

the patient calorie intake data represents a calorie intake greater than a third upper calorie intake threshold; or

the patient satiety score is less than a third lower patient satiety threshold.

The digital therapeutic program may manage the patient weight loss and the treatment regimen by:
decreasing a GLP1 dosage of the treatment regimen if:

the patient weight loss data represents a weight loss greater than an acceptable weight loss trajectory; or

the patient side effect data is representative of a level of side effects greater than a side effect intolerance threshold.

The digital therapeutic program may manage the patient weight loss and the treatment regimen by:
decreasing a calorie intake allowance of the calorie intake regimen if:

the patient calorie intake data represents a calorie intake less than a first lower calorie intake threshold;
the patient motivation score is greater than a motivation score threshold; or
the patient satiety score is greater than a first upper patient satiety threshold.

The digital therapeutic program may manage the patient weight loss and the treatment regimen by:
receiving patient weight loss data; and
setting a physical activity regimen based on the patient weight loss data.

The method may comprise setting the physical activity regimen if the weight loss data indicates a weight loss satisfying a weight loss milestone.

The method may comprise setting an intensity of the physical activity regimen setting intensity based on the patient weight loss data.

According to a sixth aspect of the present invention, there may be provided an apparatus comprising one or more processors and computer readable memory including instructions which when executed by the one or more processors carry out any method disclosed herein.

The present invention can provide advantages to patients, particularly in terms of medication compliance and their actual and perceived health. It also provides benefits to healthcare providers by providing treatment regimens in cases in which a variety of factors may determine the suitability of a therapy. For examples, it is known that both CBT and exercise therapy are helpful in managing chronic pain, as are certain drugs such as neuromodulators (for instance tricyclic antidepressants), and that the timing and that duration of each is ideally coordinated with the other.

The claimed methods and system may also be of benefit to highly variable practices in clinical care, particularly in “complex” or “at risk” patients where traditionally prescribed medication may potentially result in toxicity or suboptimal therapy. The present invention may also alleviate problems associated with a healthcare provider relying upon previous experience to personalise a treatment therapy in complex situations that lack specific dosing recommendations.

A particular advantage of the present invention is its value in the dosing of non-drug therapies, such as behavioural interventions. A drug typically has a reasonably predictable translation in efficacy from its pharmacodynamic response on the day it is given in clinic, i.e. an understood effect within 3 days or 3 weeks later when taken at home. Prior to the present invention, the way a patient interprets and responds to a non-drug intervention, for example a behavioural therapy, may vary depending on how this treatment is delivered and by whom. For instance, it may be affected by whether it is delivered by a clinician at a clinic, at a later time point (treatment/motivational fade) or if delivered electronically from day-to-day, hour-to-hour or even minute-to-minute. It may also depend upon the mental state, location and context, history and/or time-points of other drug and non-drug therapies, inter alia, as well as the amount and time of therapy delivery.

Other features and advantages of all aspects of the present invention will become apparent from the following detailed description of the invention which, when taken in combination with the accompanying drawings and examples, illustrate the principle aspects of the invention.

### BRIEF DESCRIPTION OF THE DRAWINGS

FIG. 1 shows a system suitable for implementing any of the methods described in this specification in accordance with an embodiment of the invention.

FIG. 2 shows a method that can be performed by one or more components of the system of FIG. 1 in accordance with an embodiment of the invention.

FIG. 3 shows a method that can be performed by one or more components of the system of FIG. 1 in accordance with another embodiment of the invention.

FIG. 4 shows a method that can be performed by one or more components of the system of FIG. 1 in accordance with a further embodiment of the invention.

FIG. 5 illustrates a method for treating obesity in a patient according to an embodiment of the present invention.

FIG. 6 illustrates a method of adjusting the treatment during the initial treatment titration phase according to an embodiment of the present invention.

FIG. 7 illustrates a method of achieving a stable co-therapy regimen for the progressive weight loss phase according to an embodiment of the present invention.

FIG. 8 illustrates a method of achieving a stable co-therapy regimen for the weight maintenance phase according to an embodiment of the present invention.

FIG. 9 illustrates a method of withdrawing GLP1 agonist from a co-therapy regimen for the drug withdrawal phase according to an embodiment of the present invention.

FIG. 10 illustrates a state diagram for the GLP1 co-therapy according to an embodiment of the present invention.

FIG. 11 illustrates the phased weight loss co-therapy in relation to an expected weight loss trajectory according to an embodiment of the present invention.

### DETAILED DESCRIPTION

The present invention provides personalised medicine to a patient, in particular to treat a disease or condition from which the patient is suffering. The personalised medicine may be provided in the form of co-therapy which may include one or more pharmacological therapies and/or one or more non-pharmacological therapies.

The method of the first aspect of the invention may be used to generate, or produce, a personalised medicine for a patient. The personalised medicine comprises a co-therapy regime that is suitable for use in the treatment of a disease or condition from which the patient is suffering.

As used herein, a “co-therapy regimen”, “regimen for the co-therapy” or any similar term, is a course of two or more (i.e. at least two) therapies that are to be administered to the patient with the intention of treating a disease or condition. The method may comprise two, three, four, five, six, seven or more different therapies. The regimen may comprise an associated amount, intensity and/or frequency (which may be relative to the administration of a previous dosage or to the time of day) for each of the therapies individually. Preferably the co-therapy comprises different therapies. This means that it is preferable that the therapies are not the same type of therapy, for instance, they are not two types of drugs both acting on the opiate pathway aimed at treating the same disease or condition. It is particularly preferred that the therapies do not have the same mode of action on the patient. For instance, in this particular case, while a co-therapy may provide inflammatory relief, the co-therapy may not comprise two NSAID therapies. In a particular embodiment, the co-therapies are not all hormone-based therapies.

Said therapies may be administered sequentially or concomitantly and by any route, with administration intervals between the same and/or different therapies forming part of the co-therapy regimen. For example, the co-therapy regimen may require two therapies to be administered to the patient sequentially each day or every other day. Alternatively, one or more of the therapies may be administered as required by the patient (i.e. “on demand”), or at a time at which data relating to the patient indicates that a therapy should be administered. The skilled person will know and understand the range of therapies that may be administered to a patient suffering from a particular disease or condition.

The term “one or more” means that there must be at least one of whatever follows said term, such as one, two, three, four, five six, seven, eight, nine, ten, or more.

A “therapy” may be based upon a pharmacological therapy, such as a pharmaceutical drug therapy, or a non-pharmacological therapy, such as cognitive behavioural therapy (CBT), light therapy, exercise therapy, hypnosis, massage, reflexology, and meditation. As such, the term “therapy” is to be interpreted broadly and includes any course of action that is, or may be, suitable for use in the treatment of a disease or condition.

The term “cognitive behavioural therapy” is any therapy which influences or alters the way a patient thinks and/or behaves. Its prototypical form is as a ‘talking therapy’ for mental health problems such as anxiety and depression, but it is understood in the art that similar approaches of altering the way a patient thinks and behaves is applicable to multiple other conditions, such as chronic pain, functional disorders, COPD and diabetes, such as how a patient interprets symptoms, how they interpret their interaction with the world and the future, how they control their attention; then how they behave, such as sleep/wake cycles, exercise patterns and diet. CBT may also help patients deal with overwhelming problems in a more positive way by breaking them down into smaller parts. CBT is based, in part, on the concept that a patient's thoughts, feelings, physical sensations and actions are interconnected, and that negative thoughts and feelings can cause or exacerbate certain diseases and conditions. CBT has been well documented in the treatment of depression, anxiety, obsessive compulsive disorder, panic disorder, post-traumatic stress disorder, phobias, eating disorders, such as anorexia and bulimia, sleep problems such as insomnia (in which case it may be referred to as cognitive behavioural therapy for insomnia (CBTi)), and problems associated with drug and alcohol misuse. CBT may also be used for treatment of patients with long-term health conditions such as chronic pain, COPD, diabetes, headaches, irritable bowel syndrome (IBS) and fatigue states such as chronic fatigue syndrome (CFS). Whilst it is generally believed that CBT alone cannot cure physical symptoms of IBS and CFS, it may help people cope better with their symptoms.

The term “administered” is one of the art and means that a therapy is provided, or given, to the patient. In relation to the present invention, it may be immaterial how the therapies are administered to the patient. For instance, a therapy may be administered to a patient by a healthcare provider or another third-party. The therapy may be administered by an electronic device, such as a smartphone or other handheld device, either automatically or in direct response to user input from the patient, a healthcare provider or another third-party. Alternatively, the patient may administer the therapy himself or herself, such as by taking tablets or meditating. The electronic device may act on instructions provided by a second electronic device that is located remotely from the electronic device, such as a Cloud-based server, where such instructions are transmitted to the electronic device over a network, e.g. the internet or a cellular network.

When a therapy is a pharmacological therapy, any suitable route may be used to administer said therapy. Preferably the route of administration is by oral, rectal, nasal, topical (including buccal and sublingual), transdermal, intrathecal, transmucosal or parenteral (including subcutaneous, intramuscular, intravenous and intradermal) administration. Pharmaceutical compositions useful in a pharmacological therapy may be formulated in unit dosage form, for instance in tablets and sustained release capsules, and in liposomes. Alternatively, pharmaceutical compositions may be provided as un-dosed gels, liquids and syrups to be dosed (by the patient, third-party or an automatic dosage device) prior to administration. Dosage forms useful in relation to the present invention may be prepared by any methods well known in the art of pharmacy. It is envisaged and preferable that a dosage form may be provided in a “smart pack”, i.e. a device that monitors the administration of a medicament to a patient. Such smart packs may be used to provide data to the patient and/or a third-party (for instance, a healthcare provider) on the patient's compliance with the co-therapy regimen. Said data may also be used in the present invention to modify the dataset as outlined below.

It may be the case that a patient has access only to unit dosage forms containing certain amounts of active pharmaceutical ingredients. If this is the case, the co-therapy generated in the first aspect of the invention may “round” the dosage regimen in increments that are available to the patient. For instance, if the patient has access to unit dosages comprising 0.2 mg and 0.5 mg of melatonin, then the method may generate a co-therapy regimen that is limited to increments of 0.2 mg and 0.5 mg of melatonin, for instance 0.2, 0.5, 0.7, 0.9, 1.0, 1.2, 1.4, 1.5, 1.7, 1.9 and 2.0 mg of melatonin.

When a therapy is a non-pharmacological therapy, any suitable route may be used to administer said therapy. Preferably a non-pharmacological therapy is administered by an electronic device, such as a computer, a smartphone or another handheld device. The non-pharmacological therapy may be administered either automatically or in direct response to user input from the patient, a healthcare provider or another third-party. The electronic device may act on instructions provided by a second electronic device that is located remotely from the electronic device, such as a Cloud-based server, where such instructions are transmitted to the electronic device over a network, e.g. the internet or a cellular network.

The term “treatment” includes the amelioration of the disease or condition, or a symptom or symptoms thereof. Treatment also includes the amelioration of the side-effects of another therapy, such as a pharmacological therapy. Treatment also includes the reduction in a patient's dependence on another pharmacological drug, or behaviour. “Amelioration” is an improvement, or perceived improvement, in the patient's condition, or a change in a patient's condition that makes it, or side-effects, increasingly tolerable.

In relation to a patient suffering from a disease or condition, the term “suffering” includes the patient having the disease or condition. It also covers patients expecting to suffer from the disease or condition, for instance when the method is used as a preventative measure.

The term “comprises”, and variations thereof, do not have a limiting meaning where these terms appear in the description and claims. Such terms will be understood to imply the inclusion of a stated step or element or group of steps or elements but not the exclusion of any other step or element or group of steps or elements.

The term “consisting of” means including, and limited to, whatever follows the phrase “consisting of”. Thus, the phrase “consisting of” indicates that the listed elements are required or mandatory, and that no other elements may be present for that particular feature.

The term ‘the Cloud’, or equivalently ‘Cloud-based’, should be understood to be a reference to one or more configurable computing resources that can be called upon to perform tasks according to need. The computing resources are located remotely from a user or a data processing device associated with the user and are accessible over a network such as the internet or a cellular network.

The term ‘machine-readable’ means in a format that is processable by a data processing device. Processing includes but is not limited to one or more of: identifying and displaying one or more data items stored in a machine-readable data structure on a display device; and extracting one or more data items stored in a machine-readable data structure and performing one or more calculations on said data items.

Step (a) of the first aspect of the invention involves establishing a desired patient endpoint. The desired patient endpoint may be a goal that is expected to be achieved by the administration of the co-therapy regimen to the patient. The desired patient endpoint may be set by a healthcare provider, the patient, or a combination thereof. The desired patient outcome may be specific to the particular disease or condition, and/or the patient. It may be a single goal, or a group of goals. The endpoint may be that which forms the optimal balance between beneficial effects of the therapy and side effects, as determined by the clinician, the patient, or ideally both.

The desired patient endpoint should be represented and stored in a manner such that it accessible to, and readable by, a data processing device so that it may be used in processing step (d). The desired patient endpoint can be stored either by the patient or by a healthcare provider. It will be understood that storing can thus include entering the desired patient endpoint into a data processing device using a user interface thereof, where the data processing device creates a machine-readable representation of the desired patient endpoint and stores this representation in a non-volatile storage medium or media. The non-volatile storage medium or media are preferably Cloud-based, and accessible via a network such as the internet or a cellular network.

The machine-readable representation of the desired patient endpoint may be stored in a structured format such as an element in a database or a semi-structured format such as an element node in an XML document. The representation of the desired patient endpoint may comprise one or more data types, including but not limited to one or more strings, integers, double precision values, floating point values, Boolean values, and combinations thereof. A suitable representation for a desired patient endpoint will be determined by a skilled person given the particular circumstances of any specific scenario.

Preferably, the machine-readable representation of the desired patient endpoint is stored securely, to protect patient confidentiality. It is preferably necessary to supply one or more authorisation credentials to gain access to the stored representation of the desired patient endpoint. The representation of the desired patient endpoint may additionally or alternatively be stored in an encrypted format. Such techniques are known per se and accordingly are not described in detail here.

The desired patient endpoint may comprise the successful treatment of a disease or condition so that the patient no longer suffers from said disease or condition or a symptom thereof. It may include amelioration of a side-effect of a disease or condition, a side-effect attributed to a pharmaceutical drug, such as one administered to the patient to treat a disease or condition, and/or amelioration of the side-effects of a non-pharmacological therapy. In this case, the patient may have input in defining the desired patient endpoint in relation to the tolerable side-effects or the like. The desired patient endpoint may be the patient achieving a specific value on a known symptom scale, such as a pain value as defined by the Wong-Baker Faces Pain Scale.

The desired patient endpoint may be heavily dependent upon the disease or condition to be treated. If insomnia is the disease or condition, then the desired patient endpoint may comprise the patient having at least about 4 hours sleep, for instance at least about 5 hours sleep, preferably at least about 6 hours sleep, more preferably at least about 7 hours sleep in a 24 hour period, and preferably at night. It may comprise the patient not waking before a certain time in the morning, such as not before about 7 am, for instance not before about 6 am, preferably not before about 5 am, and/or not experiencing difficulty falling asleep. The desired patient endpoint may be that the patient feels that he or she has been getting enough quality sleep over the course of a 5 day period.

If diabetes is the disease or condition, then the desired patient endpoint may comprise the patient having a blood glucose level below a certain amount, such as from about 4.0 to about 7.0 mmol/L pre-prandial, preferably from about 4.0 to about 5.9 mmol/L pre-prandial. The desired patient endpoint may comprise the treatment or amelioration of a side-effect of having diabetes, such as fatigue or burning sensations in feet. It may comprise reducing the side-effects due to medicine used to treat diabetes, such as metformin related gut side effects.

If hypertension is the disease or condition, then the desired patient endpoint may comprise the patient having a blood pressure at rest of from about 110 to about 130 mmHg systolic, and optionally having from about 60 to about 85 mmHg diastolic. It may comprise a reduction in symptoms associated with hypertension, such as reduction in headaches. Conversely, it may be desired to minimise the frequency of symptoms related to postural hypotension, such as light headedness, in those patients who have periods during the day of lower blood pressure, recognising that blood pressure often varies in any one individual during the day and from day to day. The reduction in said symptoms may be the reduction to a certain frequency of incidence.

If opiate dependency is the disease or condition, then the desired patient endpoint may comprise the patient discontinuing use of opiates. It may comprise the reduction in tolerance to opioids and/or reducing or ameliorating withdrawal symptoms, for instance nausea, diarrhoea, trouble sleeping/insomnia, jitteriness, sweating, pain recrudescence or low mood.

It is envisaged that the desired patient endpoint may change as treatment of the disease or condition progresses. For instance, the patient and/or healthcare provider may decide that that endpoint may not be attainable, or that there is no need to reach said endpoint. For instance, the desired patient endpoint for insomnia may be that the patient has 7 hours sleep in a 24 hour period, however, a patient may feel that 6 hours sleep in a 24 hour period is adequate. In this case, the desired patient endpoint may be changed accordingly. Similarly, if the desired endpoint is that the patient has 6 hours sleep in a 24 hour period, and the patient feels that 7 hours sleep in a 24 hour period is achievable, then the desired patent endpoint may be changed accordingly.

Step (b) of the first aspect of the invention involves identifying the patient position relative to the desired patient endpoint. The patient position should be related to desired patient endpoint. For instance, if the desired patient endpoint is the patient having a blood pressure at rest of from about 110 to about 130 mmHg systolic, then the patient position may be the patient's current resting blood pressure. If the desired patient endpoint is the patient having a blood glucose level of between about 4.0 to about 5.9 mmol/L pre-prandial, then the patient position may be the patient's current blood glucose level pre-prandial.

The difference between the patient position and the desired patient endpoint may be used to define the scope of treatment expected to be delivered by the co-therapy regimen. For example, if the desired patient endpoint is the patient having a blood pressure at rest of from about 110 to about 130 mmHg systolic, and the patient's current resting blood pressure is about 170 mmHg systolic, then the aim of the treatment would to reduce the patient's resting blood pressure by about 40 mmHg to about 50 mmHg. In the circumstance that the patient's average blood pressure was 125 mmHg, but at times during a day it could be 115 mmHg or 145 mmHg, and when 115 mmHg this was associated with troublesome dizziness, but if a mean blood pressure of 135 mmHg is the target, no periods of symptomatic low blood pressure occur, then a target of 135 mmHg may be preferred by both patient and clinician.

In a further variation, the initial target may be so adjusted to a systolic of 135 mmHg, but with the passage of further time, for example 3 months, the patient's vasculature may adapt such they are now able to tolerate a systolic of 115 mmHg and therefore the new average systolic target is 125 mmHg.

The patient position relative to the desired patient endpoint should be stored in the same manner as described above in respect of the desired patient endpoint so that it may be used in processing step (d). Preferably, the machine-readable representation of the patient position is stored in the same format as the machine-readable representation of the desired patient endpoint, as this may enable or assist with calculation of the difference between the patient position and the desired patient endpoint by a data processing device.

Step (c) of the first aspect of the invention involves generating or modifying a dataset relating to the patient, based on one or more patient-related measurements.

As used herein, a “dataset” is a machine-readable collection of information, or data, that is composed of separate elements, which elements can be manipulated either individually or collectively by a processor, such as a processor in a data processing device. The information, or data, in the dataset is related to the patient. A dataset can take many forms including a structured, semi-structured or unstructured dataset.

Without being bound by theory, it is understood that patient-related measurements are those that are expected to be useful in generating a co-therapy regimen suitable for use in the treatment of the disease or condition to which the method relates. The dataset may therefore help to predict the patient's susceptibility to treatment of a particular disease with the co-therapy.

A combination of the patient's susceptibility to treatment and the scope of treatment, defined by the patient position relative to the desired patient endpoint, may be use to provide the co-therapy regime.

It is envisaged that the claimed method may be used to help predict the efficacy and/or suitability of a co-therapy regime for a particular patient.

If a dataset relating to the patient is not available, perhaps because the patient has just enrolled onto the system, then said dataset is generated. This involves creating a dataset containing the relevant data as discussed below, and linking the dataset to the patient, such as by using a patient code or some other unique identifier. If a suitable dataset relating to the patient is already available to the data processing device, then the dataset may be modified by appending one or more relevant data entries to the dataset and/or replacing one or more relevant data entries already present in the dataset, said data being that as discussed below.

The patient-related measurement may include
a) one or more physiological measurements;
b) one or more patient-centred outcomes;
c) one or more environmental measurements, such as temperature, humidity, and/or light intensity, local to the patient; and/or
d) one or more behavioural factor measurements.

The term “patient-related measurement” refers to data that is related to the patient. A patient-related measurement may be data relating directly to the patient, such as a “physiological measurement”, e.g. resting heart rate, systolic blood pressure at rest, blood glucose level, and biomarker concentration in blood. Such measurements may be taken by a patient, a healthcare provider or by a device, such as an electronic device, such as a smartphone or other handheld device. In any case, the one or more patient-related measurements used in the method of the invention may be dependent upon the specific disease or condition to be treated.

A patient-related measurement may be a patient-centred outcome.

A “patient-centred outcome” is an assessment of the patient's beliefs, opinions and needs, optionally in conjunction with a healthcare provider's expertise, in relation to their treatment. A patient-centred outcome may comprise an indication of whether the patient is receiving suitable relief from one or more symptoms of the disease or condition from which they are being treated. For instance, it could be an indication of whether the treatment is providing enough pain relief, or enough sleep, to the patient. It may also comprise negative effects of the treatment including various side effects and the preferred trade-off between beneficial effects and side effects of the treatment.

A patient-centred outcome may be reported and/or recorded by the patient, a healthcare provider, or an electronic device, such as a smartphone or other handheld device. If the patient-centred outcome is reported and/or recorded by the patient it may be referred to as a patient-reported outcome.

A patient-centred outcome, and therefore a patient-reported outcome, may be qualitative or quantitative. A patient-centred outcome, and especially a patient-reported outcome, may need to be mapped onto a predefined scale to create a mapped patient-centred outcome. This is preferable if the patient-centred outcome (or patient-reported outcome) is qualitative. It may be preferable that a patient-reported outcome is provided via a questionnaire. Further, the particular predefined scale may be personalised for the patient.

A patient-related measurement may relate to the patient's environment, such as the patient's local environment. In this case, an environmental measurement may be made. Suitable environmental measurements may include temperature, humidity, and/or light intensity, such as daily light exposure, daily average temperature, maximum/minimum daily temperature, and daily rainfall. The environmental measurement may be reported and/or recorded by the patient, a healthcare provider, or an electronic device, such as a smartphone or other handheld device.

A patient-related measurement may be a behavioural factor measurement. These are measurements of specific behaviours of the patient, such as total number of steps taken per day, minutes of cardiovascular training performed per day, and units of alcohol consumed per week. A behavioural factor measurement may be qualitative or quantitative. A behavioural factor measurement may need to be mapped onto a predefined scale to create a mapped behavioural factor measurement. This is preferable if the behavioural factor measurement is qualitative.

As mentioned, patient-related measurements useful in the method of the present invention may be dependent upon the disease or condition that it intended to be treated. Again, the patient-related measurements that may be expected to be useful in generating a co-therapy regimen suitable for use in the treatment of the disease or condition will be known.

The data may be inputted directly into the dataset in raw form, or may be processed prior to being input into the dataset. Such processing may involve taking the data and modifying or evaluating one or more of the data's constituent data points prior to inputting it into the dataset. For example, the patient may provide information in the form of a patient-centred outcome, such as the level of pain he or she is experiencing by pointing to a face on the Wong-Baker Faces Pain Scale, which is then converted into a numeric value according to that scale, with the numeric value being input into the dataset.

Patient-related measurements may be taken by the patient, and/or by a healthcare provider. For instance, the patient may take their own blood pressure, heart rate, or to blood glucose level. Alternatively, a healthcare provider may take the patient's blood pressure, heart rate, or blood glucose level. A patient-related measurement may require input from multiple people. For example, the patient may provide a blood sample at a specific time, which is then analysed for a certain biomarker concentration, said concentration then inputted into the dataset. The measurements may be actively obtained, for instance when the patient and/or healthcare provider make an action specifically to obtain a measurement, such as providing a blood sample at a specific time. The measurements may be obtained passively, such as via a wearable technology, preferably linked to an electronic device, such as a smartphone or other handheld device.

Patient-related measurements may be obtained from other sources, such as online databases or third-parties. For example, data may be taken from online weather websites to estimate the daily light exposure for the patient based upon the patient's location.

It will be apparent that while each of the patient-related measurements used in the method of the invention should be expected to influence the co-therapy regimen, the level of influence may be dependent upon the specific patient-related measurement, the patient and/or the disease or condition to which the method relates. It is therefore envisaged that the step of generating or modifying the dataset relating to the patient may include the step of applying a weighting factor to each of the one or more patient-related measurements.

Step (d) of the first aspect of the invention involves processing the dataset, the patient position and the desired patient endpoint, to generate the co-therapy regimen. The processing may be carried out using a processor, such as a processor in a data processing device.

Without being bound by theory, the difference between the patient position and the desired patient endpoint may define the scope of treatment to be delivered to the patient, and the dataset may be used to predict the susceptibility of the patient to the treatment. The dataset, the patient position and the desired patient endpoint may therefore be processed to provide a patient-specific dosage regimen (personalised medicine) that is expected to treat the particular disease or condition.

In the processing step, the dataset, the patient position and the desired patient endpoint may be processed using a rules-based system to produce the regimen for the co-therapy. Alternatively, the dataset, the patient position and the desired patient endpoint may be processed using one or more machine learning algorithms to produce the regimen for the co-therapy. As a further alternative, a hybrid approach is also contemplated in which both a rules-based system and one or more machine learning algorithms are used to process the dataset, patient position and desired patient endpoint.

The term “rules-based system” means a system that operates according to a set of one or more predefined rules. The one or more rules may be encoded in a computer-interpretable format such as one or more modules of program code. The one or more rules may be encoded to take advantage of known or hypothesised relationships between a patient position and their desired patient endpoint, and/or observations in changes in the behaviour, health or other such parameters of the patient as the treatment progresses, in order to produce a recommended regimen for the co-therapy. Other factors not relating to the condition of the patient, such as regulatory constraints, may be additionally or alternatively encoded into the one or more rules.

One or more of the rules may be modified or deleted to take account of new observations, hypotheses and/or knowledge as and when appropriate. One or more new rules may be added to an existing set of one or more rules, with the one or more new rules perhaps being introduced to take account of new observations, hypotheses and/or knowledge, and/or changes in a regulatory framework.

A rule may reference another entity such as the above-discussed dataset. A rule may specify that a particular action is or is not taken based on a property of the entity; e.g. a value of a data point in the dataset. A rule may involve instructing a data processing device to perform a calculation, which calculation perhaps involves or is based upon a property of the entity, where an action being performed as an outcome of the rule depends on an output of the calculation. A rule may reference one or more external sources of data, such as a database of a medical institution, with the action specified by the rule being dependent on data retrieved from said database.

The term “machine learning algorithm” takes its usual meaning in the art and includes any algorithm that employs any currently known or later developed machine learning technique or techniques. Examples of machine learning algorithms include but are not limited to a neural network, a support vector machine, a Naïve Bayes Classifier, a K-Means Clustering Algorithm, and the like. Deep learning techniques may be used.

The machine learning algorithm may employ supervised, semi-supervised and/or unsupervised learning techniques.

In the context of the present disclosure, the at least one machine learning algorithm is used either alongside or in place of the above-discussed rules-based system and has the objective of producing the regimen for the co-therapy. The one or more machine learning algorithms may use one or more data points from the above-discussed dataset for inputting into a model, where the output of the model is a regimen for the co-therapy. The model may be trained using one or more data points from the above-discussed dataset. Training of a machine learning model, and use of a trained model, are known per se in the art and thus are not discussed in detail here.

The co-therapy may comprise pharmacological therapies, non-pharmacological therapies or a mixture thereof. Specifically, the co-therapy used in the methods of the invention may comprise
(i) two or more pharmacological therapies;
(ii) one or more pharmacological therapies and one or more non-pharmacological therapies, preferably wherein the one or more non-pharmacological therapy is cognitive behavioural therapy; or
(iii) two non-pharmacological therapies, preferably wherein at least one non-pharmacological therapy is cognitive behavioural therapy.

In a second aspect of the invention, there is provided a method of treating a patient suffering from a disease or condition, the method comprising the steps of
a) selecting a co-therapy suitable to treat the disease or condition;
b) establishing a desired patient endpoint;
identifying the patient position relative to the desired patient endpoint;
d) generating or modifying a dataset relating to the patient, based on one or more patient-related measurements;
e) processing the dataset, the patient position and the desired patient endpoint to produce a regimen for the co-therapy; and
f) administering the co-therapy to the patient according to the regimen.

As it will be appreciated, steps (b) to (e) of the second aspect of the invention correspond to steps (a) to (d) of the first aspect of the invention, the definition of which for the first aspect of the invention applies equally to the second aspect of the invention.

In addition to the above, step (a) of the second aspect of the invention involves selecting a co-therapy suitable to treat the disease or condition. The range of co-therapies that may be suitable for use in the treatment of a specific disease or condition are known, particularly to healthcare providers.

Step (f) of the second aspect of the invention involves administering the co-therapy to the patient according to the regimen. As mentioned above, it is within the scope of this invention that the co-therapy is administered to the patient according to the regimen in any suitable way.

It is envisaged that successful treatment of the disease or condition may require a plurality (i.e. more than one) of treatment cycles. A treatment cycle can comprise each of steps (a) to (f) of the second aspect of the invention. Therefore, the method of treatment according to the second aspect of the invention may include carrying out steps (a) to (f) and then repeating steps (a) to (f) at least one time, such as one, two, three, four, five, six, seven, eight, nine, ten times.

In the method of treatment according to the second aspect of the invention, it is preferable not to change the co-therapy between treatment cycles. Therefore, it is preferable that the method comprises a plurality of treatment cycles, wherein the treatment cycle comprises steps (b) to (f).

When a plurality of treatment cycles is to be used, the frequency of the cycles may be dependent upon the specific therapies being administered to the patient. In particular, the time period between processing step (e) of the second aspect of the invention (i.e. the step of processing the dataset, the patient position and the desired patient endpoint to produce a regimen for the co-therapy) of two consecutive cycles may be dependent upon the timeframe within which one would expect the patient to respond to the therapies. For example, if the patient is expected to have a fast response time to a treatment, such as the use of insulin to treat diabetes, processing step (e) may be carried out at least about 1 hour, such as at least about 2 hours, for instance at least about 3 hours, for examples at least about 4 hours, after processing step (e) was last performed. If the patient is expected to have an intermediate response time to a treatment, such as the use of melatonin to treat insomnia, processing step (e) may be carried out at least about 1 day, such as at least about 2 days, for instance at least about 3 days, for examples at least about 4 days, after processing step (e) was last performed. If the patient is expected to have a slow response time to a treatment, such as the use of cognitive behavioural therapy in the treatment of opioid dependence, processing step (e) may be carried out at least about 1 week, such as at least about 2 weeks, for instance at least about 3 weeks, for examples at least about 4 weeks, after processing step (e) was last performed.

Notwithstanding the above, a benefit of the present invention is that a patient's co-therapy regimen may be altered within a shorter time period than that set by two consecutive visits to a healthcare provider (the first visit providing the patient with a co-therapy regimen and the second visit altering the co-therapy regimen based upon the patient's response to the co-therapy regimen). The frequency of visits to a healthcare provider may be dependent upon the type of therapies being administered to the patient, therefore, it is preferable that the time period between processing step (e) of the second aspect of the invention of two consecutive cycles is less than the frequency of such visits. For instance, processing step (e) may be carried out less than about 10 weeks, such as less than about 8 weeks, for instance less than about 6 weeks, for examples less than about 5 weeks, after processing step (e) was last performed. In certain cases, processing step (e) may be carried out less than about 4 weeks, such as less than about 2 weeks, for instance less than about 1 week, for examples less than about 4 days, i.e. less than about 2 days, after processing step (e) was last performed.

In an exemplary embodiment, in step (d) the dataset may be modified based on one or more patient-related measurements. However, step (e) is not performed until an elapsed time has passed, wherein the elapsed time is equal to a length of time between the production of a regimen for the co-therapy according to step (e) and the most recent modification to the dataset in step (d). Essentially, step (d) may be carried out a plurality of times until a certain time has elapsed since the production of a regimen for the co-therapy in step (e) has occurred, after which step (e) is performed. The elapsed time may be the time noted above between processing step (e) of two consecutive cycles.

When a plurality of treatment cycles is used, the method may comprise after step (e) an additional step of adjusting the regimen for the co-therapy based upon the difference between the regimen provided in step (e) and the regimen provided in step (e) of the previous cycle. In this case, the adjusted regimen for the co-therapy may be adjusted by 60, 70, 80, or 90% of that difference. For instance, when an 80% threshold is adopted, in the case when the regimen provided in the previous cycle comprises 100 mg of a drug, and the new regimen comprises 200 mg of the drug, the method may return a regimen of 180 mg to be administered to the patient in step (f). This additional step may help dampen the method's reaction to a change in the patient dataset and prevent the patient's regimen for the co-therapy oscillating between a dose of the drug that is too high and a dose that is too low to attain the desired patient endpoint.

The processing step may also limit the maximum and minimum amount that one or more drugs may be administered to the patient, and/or the amount by which the regimen for the co-therapy is changed, based upon regulatory matters, patient or healthcare provider instruction, or other factors.

Further, it may not be necessary to once again establish the desired patient endpoint so the method of the second aspect of the invention may comprise a plurality of treatment cycles, wherein the treatment cycle comprises steps (c) to (f).

Also envisaged in the present invention is a co-therapy for use in the treatment of a patient suffering from a disease or condition, the co-therapy provided by a method comprising the steps of the first aspect of the invention and all embodiments thereof.

The co-therapy of generated or provided by the methods of the present invention may be used to treat or prevent any disease or condition. This includes both acute and chronic diseases and conditions, such as those selected from the group consisting of pre-diabetes, diabetes; cardiovascular disease; neurodegeneration diseases, such as Mild Cognitive Impairment (MCI), Alzheimer's disease and Parkinson's disease; atrial fibrillation; attention deficit hyperactivity disorder (ADHD); autoimmune diseases, such as ulcerative colitis, lupus erythematosus, Crohn's disease, coeliac disease, Hashimoto's thyroiditis, bipolar disorder; cerebral palsy such as dyskinetic and athetoid; chronic graft-versus-host disease; hepatitis; chronic kidney disease; arthritis and chronic osteoarticular diseases, such as osteoarthritis and rheumatoid arthritis; cancer; obesity; asthma; sinusitis; cystic fibrosis; tuberculosis; chronic obstructive airways disease, bronchitis; bronchiolitis; pulmonary fibrosis; pain, including chronic pain syndromes; depression; eating disorders; polycystic ovary syndrome; epilepsy; fibromyalgia; viral diseases, such as HIV/AIDS; Huntington's disease; hypotension; hypertension; allergic rhinitis; multiple sclerosis; fatigue states, including chronic fatigue syndrome; insomnia; narcolepsy; osteoporosis; periodontal disease; postural orthostatic tachycardia syndrome; sickle cell anaemia and other haemoglobin disorders; sleep apnoea; thyroid disease; reflux, including gastroesophageal reflux; vomiting; irritable bowel syndrome (IBS); inflammatory bowel disease (IBD); peptic ulcer; acute urticarial; atopic dermatitis; contact dermatitis; seborrheic dermatitis; headache, including migraine, cluster headache, and tension-type headache; addiction, such as drug addiction, in particular opiate dependency, cocaine, alcohol, or nicotine addiction and chronic usage thereof; thromboembolic disease; hair loss; hormone replacement therapy; psychiatric disorders, such as psychosis, anxiety and depression; endocrine dysfunctions, including growth hormone deficiency, hypothyroidism; haematological disorders, including clotting factor deficiencies or low levels of white or red blood cells; neurodevelopmental delay (NDD) disorders, including Autistic Spectrum Disorder (ASD), Smith Magenis Syndrome and ADHD; parasomnias, including REM and NREM parasomnias and nightmare disorders; sleep movement disorders, such as restless legs syndrome and periodic limb movement disorder, circadian rhythm disorders (including such disorders brought on by shift work and/or jet lag); chorea and tic disorders.

Diseases and conditions in which the present invention is particular useful are insomnia, obesity, diabetes, in particular type-II diabetes, hypertension, and opiate dependency.

Data collected when the disease or condition is insomnia may relate to one or more of the possible data for insomnia in the table below.

Possible data for insomnia

Melatonin (current dosage regimen)
Melatonin metabolite levels in urine at various time points
Daily light exposure
CBT usage
Sleep quality
Tiredness level
Perceived impairment of ability
Caffeine intake
Pain
Anxiety
Stress
Insomnia duration (from start of suffering)
Basal heart rate
Metabolic rate
Nocturnal cognitive arousal, such as determined by non-invasive brain
wave (EEG) recordings
Multiple sleep latency test results
Underlying medical conditions/patient history
Oxygen haemoglobin desaturation during REM sleep
Other medication
Gender
Age
Ethnicity
Smoking history
Alcohol use
Illicit substance use
Nocturnal Polysomnogram
Actigraphy
Daytime Sleepiness
Depression
Oximetry
Chronotype
Maintenance of Wakefulness Test
Arousal Index
Any other sleep disorder (e.g. presence of restless legs syndrome)
Nocturnal panic
Pain
Night sweats

Data collected when the disease or condition is type-II diabetes may relate to one or more of the possible data for type-II diabetes in the table below.

Possible data for type-II diabetes

Metformin usage (current and historic dosage regimen)
GLP-1 agonist usage (current and historic dosage regimen)
Insulin usage (current and historic dosage regimen)
Other diabetes medication usage (current and historic dosage regimen)
Fasting blood glucose level
Current blood glucose level
Diet
CBT usage
Sleep quality
Activity levels
Fatigue levels
Energy levels
Weight
Mood
Basal heart rate
Metabolic rate
Underlying medical conditions/patient history
Other medication
Gender
Age
Ethnicity
Smoking history
Alcohol use
Bowel function (inc. degree of bloating and cramping, bowel openings,
looseness of stool, nausea degree)
Timing of symptoms
Gut microbiome analysis

As mentioned above, the co-therapies suitable to treat diseases and conditions are well known. However, when the disease or condition is insomnia, it may be advantageous for the co-therapy to consist of two therapies, the first therapy comprising melatonin and the second therapy comprising cognitive behavioural therapy for insomnia (CBTi).

When the disease or condition is diabetes, particular type-II diabetes, it may be advantageous for the co-therapy to consist of two therapies, the first therapy comprising metformin and the second therapy comprising cognitive behavioural therapy.

When the disease or condition is diabetes, particular type-II diabetes, it may be advantageous for the co-therapy to consist of two therapies, the first therapy comprising metformin and the second therapy comprising a GLP-1 agonist.

When the disease or condition is diabetes, particular type-II diabetes, it may be advantageous for the co-therapy to consist of two therapies, the first therapy comprising a GLP-1 agonist and the second therapy comprising cognitive behavioural therapy.

When the disease or condition is diabetes, particular type-II diabetes, it may be advantageous for the co-therapy to consist of three therapies, the first therapy comprising metformin, the second therapy comprising cognitive behavioural therapy and the third comprising a GLP-1 agonist.

When the disease or condition is hypertension it may be advantageous for the co-therapy to consist of two therapies, the first therapy comprising amlodipine and the second therapy comprising cognitive behavioural therapy.

When the disease or condition is opiate dependency it may be advantageous for the co-therapy to consist of two or three therapies. When the co-therapy consists of two therapies, it is preferable that the first therapy comprises morphine, and the second therapy comprises an az agonist or cognitive behavioural therapy. When the co-therapy consists of three therapies, it is preferable that the first therapy comprises morphine, the second therapy comprises an az agonist, and the third co-therapy comprises cognitive behavioural therapy. In each case, it is preferable that the az agonist is clonidine.

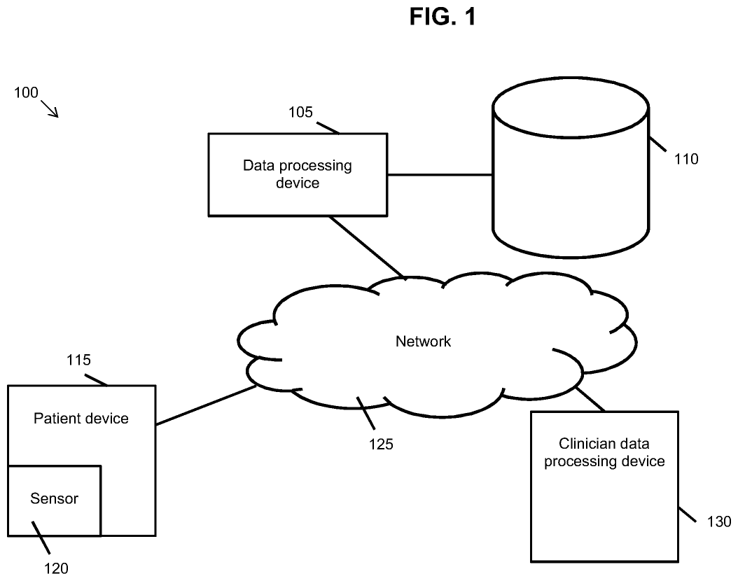

A system 100 suitable for carrying out any of the above-described methods is shown in FIG. 1. System 100 includes a data processing device 105 that is communicatively coupled to a database 110 that stores a dataset as discussed earlier in this specification. Database 110 is stored on a storage medium, e.g. a Cloud-based storage medium.

Data processing device 105 comprises at least one processor and is configured to carry out any of the methods described in this specification, or one or more steps thereof. Data processing device 105 may operate in accordance with one or more rules, optionally stored in database 110, and/or data processing device 105 may be configured to execute one or more machine learning tasks. The machine learning tasks include any combination of: training a model using data from a dataset stored in database 110 and/or using a trained model to classify an input such as data from a dataset stored in database 110.

Data processing device 105 can be configured to perform tasks including: receiving data from a patient device 115; generating a dataset for storage in database 110; appending data to an existing dataset stored in database 110; transmitting information and/or commands to patient device 115 and/or clinician data processing device 130, and the like. Data processing device 105 may be a server that hosts a website or portal which is accessible to one or both of patient device 115 and clinician data processing device 130.

In the illustrated embodiment, patient device 115 is a smartphone, optionally comprising a sensor 120. However, the invention is not limited in this respect and patient device 115 can take many other forms, including but not limited to a mobile telephone, a tablet computer, a desktop computer, a voice-activated computing system, a laptop, a gaming system, a vehicular computing system, a wearable device, a smart watch, a smart television, an internet of things device, a medicament-dispensing device and a device including a drug pump.

Patient device 115 is communicatively coupled to data processing device 105 via a network 125. In the illustrated embodiment network 125 is the internet, but the invention is not limited in this respect and network 125 could be any network that enables communication between patient device 115 and data processing device 105, such as a cellular network or a combination of the internet and a cellular network.

Patient device 115 is configured to gather data relating to a patient and/or the immediate environment of the patient and to transmit at least some of said gathered data to data processing device 105. Patient device 115 may gather data using sensor 120, which can be any combination of: a light sensor such as a camera, a temperature sensor, an acoustic sensor such as a microphone, an accelerometer, an air pressure sensor, an airborne particulate sensor, a global positioning sensor, a humidity sensor, an electric field sensor, a magnetic field sensor, a moisture sensor, an air quality sensor and a Geiger counter, and/or any other such sensor capable of determining a characteristic of the patient and/or the patient's immediate environment.

Alternatively, sensor 120 can be omitted from patient device 115. In that case, information about the patient and/or the immediate environment of the patient can be obtained via other mechanisms including manual data entry using a human interface device of patient device 115.

It will be appreciated that system 100 may include more than one patient device that is similar to patient device 115. It is contemplated that a single patient may use more than one patient device to collect data and feed it into system 100.

Patient device 115 may have one or more applications installed on a storage medium associated with the patient device (not shown), the one or more applications configured to control data acquisition via sensor 120 and/or to assist the patient in providing data relating to their current condition and/or their immediate environment.

System 100 optionally includes a clinician data processing device 130 that is communicatively coupled via network 125 to data processing device 105. The clinician data processing device 130 is broadly similar to patient device 115, offering a similar set of functionality. Specifically, the clinician data processing device 130 enables data relating to the patient and/or the immediate environment of the patient to be collated and transmitted to data processing device 105. Clinician data processing device 130 is contemplated as being physically located at a clinician's premises during its use, such as a doctor's surgery, a pharmacy or any other healthcare institution, e.g. a hospital. Clinician data processing device 130 may include one or more sensors like sensor 120, and/or be configured to control one or more separate sensors like sensor 120, which sensors are capable of gathering information about the patient and/or their local environment.

It is also contemplated that clinician data processing device 130 is typically used by a medically trained person with appropriate data security clearance, such that more advanced functionality may be available than via the patient device 115. For example, the clinician data processing device 130 may be able to access a medical history of the patient, generate a prescription for the patient, place an order for medication, etc. Access to functionality may be controlled by a security policy implemented by data processing device 105.

It is contemplated that system 100 could omit patient device 115 altogether, in which case all reporting of data to data processing device 105 is handled by clinician data processing device 130. This configuration may find particularly utility in situations where a patient is incapable of providing data to data processing device via a patient device, e.g. due to their current medical condition or non-compliance.

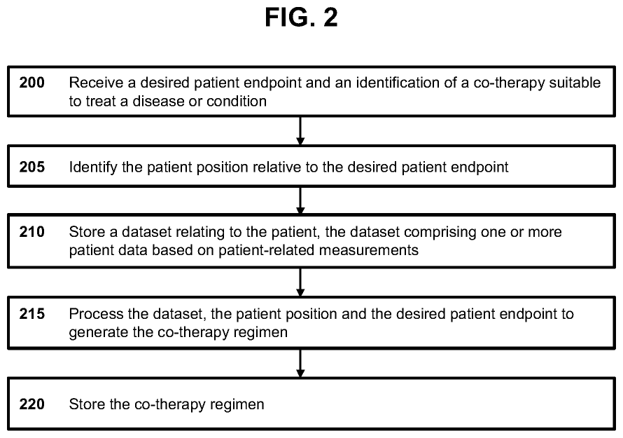

FIG. 2 shows a method that can be performed by data processing device 105 in accordance with an embodiment of the invention.

In step 200, data processing device 105 receives a desired patient endpoint. The desired patient endpoint may be received from patient device 115 or clinician data processing device 130. Data processing device 105 may store the desired patient endpoint in database 110 in a machine-processable format. The desired patient endpoint may be provided by a clinician via a user interface of clinician data processing device 130, or it may be provided by a patient via a user interface of patient device 115.

Data processing device 105 also receives in step 200 an identification of a co-therapy suitable to treat a disease or condition that the patient corresponding to the desired patient endpoint is suffering from. Data processing device 105 may store the identification in database 110 in a machine-processable format. The identification may be provided by a clinician via a user interface of clinician data processing device 130, or it may be retrieved from database 110 or another data source (e.g. a healthcare institution's database) based upon an identification of the disease or condition that the patient is suffering from, or based upon a patient unique identifier.

In step 205, data processing device 105 identifies the patient position relative to the desired patient endpoint. Data processing device 105 may receive patient-related information from one or both of the patient device 115 and the clinician data processing device 130 in order to identify the patient position.

The patient-related information includes but is not limited to: information entered by the patient using a user interface of patient device 115; data gathered by sensor 120 of patient device 115, if present; information entered by a clinician or other healthcare professional using a user interface of clinician data processing device 130; and/or data gathered by a sensor of clinician data processing device, if present.

In the case where sensor data is provided, data processing device 105 preferably identifies the patient position by processing the patient-related information using one or more rules stored in database 110 and/or using a trained machine learning model stored in database 110.

In step 210, data processing device 105 stores a dataset relating to the patient, the dataset comprising one or more patient data based on patient-related measurements. The patient-related measurements include but are not limited to: measurements entered by the patient using a user interface of patient device 115; measurements performed by sensor 120 of patient device 115, if present; measurements entered by a clinician or other healthcare professional using a user interface of clinician data processing device 130; and/or measurements performed by a sensor of clinician data processing device, if present.

In the case where a dataset relating to the patient is already present in database 110, data processing device preferably appends the patient data to this existing dataset as part of the storing operation. If no dataset relating to the patient is found in database 110, storing preferably includes creating a blank dataset, assigning a unique patient identifier to the blank dataset and populating the blank dataset with the patient data. The unique patient identifier is associated with the patient and can be generated according to any known unique identifier generation scheme.

Data processing device 105 may be configured to apply a weighting factor to each of the patient-related measurements received in step 210 when generating the patient data. The weighting factor expresses a relative importance of a particular patient-related measurement relative to other patient-related measurements. Data processing device 105 may generate an individual weighting factor for each of the patient-related measurements. A given weighting factor may have the same value or a different value to another weighting factor.

The weighting factors may be defined by a clinician in conjunction with the patient. Preferably, data processing device 105 first receives a range for each weighting factor, and subsequently receives a value for each weighting factor that is within the respective range. The selection within the range can be based upon patient preferences, such as the desire for a particular benefit and/or the level of desire to avoid a particular side effect, for example.

Preferably, each weighting factor is selected so as to minimise the time it is expected for the patient to move from the patient position to the desired patient endpoint. A probabilistic prediction of a patient condition, e.g. a Bayesian prediction, can be used to predict the future condition of the patient using current and historical patient measurements as a function of each weighting factor. The set of weighting factors is selected based on the prediction. The set of weighting factors that minimises the time it is expected for the patient to move from the patient position (i.e. their current state) to the desired patient endpoint is preferably selected. One or more weighting factors can be adjusted as necessary during the course of a treatment should the actual progress of the patient deviate significantly from the predicted progress of the patient.

In step 215, data processing device 105 processes the dataset, the patient position and the desired patient endpoint to generate a co-therapy regimen. This step can comprise processing the dataset, patient position and desired patient endpoint using one or more rules, and/or using one or more machine learning algorithms. Regardless of the technique used to generate a co-therapy regimen, the result of step 215 is a co-therapy regimen that is predicted, suggested or otherwise thought to be likely to be effective in moving the patient closer towards the desired patient endpoint.

In cases where the co-therapy includes a component requiring the patient to administer one or more drugs that are provided in a fixed dosage form, e.g. pills containing a set amount of active ingredient, step 215 preferably includes a comparison of the dosage requirements of the generated co-therapy regimen against the dosages of the relevant drug(s) that are available to the patient.

In the case where the patient cannot administer the relevant drug(s) in the amount required by the generated co-therapy regimen, data processing device 105 may adjust the generated co-therapy regimen to require an amount of the relevant drug(s) that minimises the difference between the amount required by the initially generated regimen and the possible combinations of dosages administrable by the patient.

For example, consider the case where a patient is required to administer drug X as part of a co-therapy. The patient has access to pills containing drug X, where each pill contains 10 mg of drug X. Data processing device 105 initially generates a co-therapy regimen that calls for 32 mg of drug X. The patient cannot administer precisely 32 mg, so data processing device 105 adjusts the co-therapy regimen to require 30 mg of drug X, this being administrable by the patient ingesting three 10 mg pills.

In another example, data processing device 105 initially generates a co-therapy regimen that calls for 38 mg of drug X. The patient cannot administer precisely 38 mg, so data processing device 105 adjusts the co-therapy regimen to require 40 mg of drug X, this being administrable by the patient ingesting four 10 mg pills.

Data processing device 105 can alternatively be configured to adjust the generated co-therapy regimen to require an amount of the relevant drug(s) that is equal to the closest value administrable by the patient that does not exceed the dosage initially generated by data processing device 105.

Under this alternative implementation, using the example of drug X above, in the case that the data processing device initially generates a co-therapy regimen that calls for 38 mg of drug X, the co-therapy regimen may be adjusted to require 30 mg of drug X, this being administrable by the patient ingesting three 10 mg pills. This alternative implementation may be preferred in situations where it is considered undesirable to exceed a dosage recommendation.

Information relating to the dosage forms available to the patient may be provided to data processing device 105 by patient device 115 and/or clinician data processing device 130. This information may be stored in the dataset relating to the patient as part of step 210.

As part of step 215, data processing device 105 can additionally or alternatively be configured to check whether a change in a dosage amount of one or more constituents of the co-therapy is greater than a threshold level. The threshold change can be expressed as a percentage change of the dosage amount of the most recently generated co-therapy regimen, i.e. the regimen currently being followed by the patient. The threshold level is preferably set based on a prediction as to the greatest change in dosage that a patient can safely tolerate. The threshold level may be received by data processing device 105 from a clinician, perhaps via clinician data processing device 130.

In the case that the change in dosage amount is greater than the threshold level, data processing device 105 is configured to adjust the co-therapy regimen such that the dosage amount is equal to the threshold level. This adjustment can be performed in addition to the adjustment based on dosage amounts available to the patient, or in the alternative. This adjustment has the effect of ensuring that the patient does not follow a co-therapy regimen that proscribes a change in dosage amount that is thought to be too large for the patient to tolerate.

In the case that the change in dosage amount is less than or equal to the threshold level, data processing device 105 is configured to make no adjustment to the co-therapy regimen.

In step 220, data processing device 105 stores the co-therapy regimen that was generated in step 215. The co-therapy regimen may be stored in database 110, preferably in association with the patient and more preferably in the dataset relating to the patient. Metadata such as the date and time at which the co-therapy regimen was generated may also be stored in association with the co-therapy regimen.

In the case where any adjustment to the generated co-therapy regimen of the type discussed above has been performed in step 215, an indication that this adjustment was performed may also be stored by data processing device 105 as part of step 220, e.g. within metadata associated with the co-therapy regimen. A notification may additionally or alternatively be transmitted to patient device 115 and/or clinician data processing device 130 by data processing device 105, to inform one or both parties that an adjustment to the co-therapy regimen has been made.

The timing of said transmission may be optimised based on the regiment and the most recent patient data (for example the patient's location or state of mind).

Step 220 may also comprise transmitting the co-therapy regimen to one or both of patient device 115 and clinician data processing device 130, perhaps for display on a display of one or both of these devices. Additional actions that data processing device 105 may perform as part of step 220 include any combination of: generating a prescription for the patient based on the co-therapy regimen; instructing the patient to follow the co-therapy regimen; and controlling a drug administration device to cause at least one drug associated with the co-therapy regimen to be administered to the patient. Data processing device 105 may effect these additional actions by transmitting control commands to other devices, including but not limited to patient device 115 and/or clinician data processing device 130.

Preferably, data processing device 105 is configured to make a determination as to whether the co-therapy regimen generated in step 215 is compliant with requirements, guidelines, etc. of a relevant regulatory framework. Checking for regulatory compliance may include checking that a recommended dosage of a drug that is part of the co-therapy is within a dosage range that has regulatory approval, for example. If the recommended dosage is non-compliant, remedial action by the data processing device may be taken, such as: setting a dosage of the drug to a value that has regulatory approval and which is closest to the recommended value; and/or transmitting a message to clinician data processing device 130 requesting further instructions.

Database 110 may store a regulatory data table that identifies, on a drug by drug basis, dosage ranges having regulatory approval, for use by data processing device 105 when checking that the co-therapy regimen generated in step 215 is regulatory compliant.

It will be appreciated that steps 200 to 220 can be performed by data processing device 105 a plurality of times for a single patient. In this way, a dynamic co-therapy is provided that is adaptive to the changing condition of the patient as the treatment progresses. Without being bound by theory, it is thought that the adaptation of a co-therapy as treatment progresses may result in a more effective treatment for the patient. For example, the patient may achieve, or get close to achieving, the desired patient endpoint, perhaps in a relatively rapid manner.

It will also be appreciated that in some cases it is appropriate to keep the desired patient endpoint and co-therapy constant over the course of a treatment. In such cases, for the second and subsequent iteration of the process of FIG. 2, data processing device 105 can omit step 200 as the desired patient endpoint and identification of a suitable co-therapy are unchanged.

It is contemplated that, when performing one or more steps of FIG. 2, data processing device 105 may receive one or more pieces of information in a human-intelligible format that is not suitable, or at least not optimised, for storage in the dataset stored in database 110. For example, the patient-related measurements may include patient-reported outcomes provided in the form of natural language or as values within a constrained response framework. In such cases, data processing device 105 is preferably configured to map the patient-reported outcomes onto a predefined scale to create mapped patient-reported outcomes. In this way the ‘messy’ information received by data processing device 105 can be converted into ‘clean’ data before being stored in database 110.

It is also contemplated that a variant of step 200 may be performed in the second and subsequent iteration of the process of FIG. 2, in which only the desired patient endpoint is received by data processing device 105. This variant is particularly suited for use in a case where the co-therapy remains constant but the desired patient endpoint may vary over time.

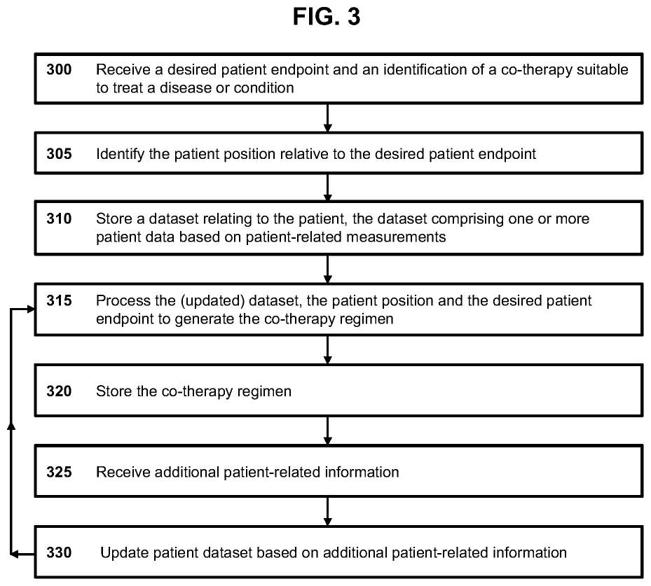

An exemplary embodiment in which data processing device 105 performs more than one iteration is shown in FIG. 3. Steps 300 to 320 are the same as steps 200 to 220, respectively, so are not described in detail again here. The following sets out additional considerations that are preferably present in an iterative process like that of FIG. 3.

In step 325, data processing device 105 receives additional patient-related information.

The patient-related information can be of the type discussed earlier in this specification and is received subsequent to the patient-related information received in connection with step 205.

In step 330, data processing device 105 updates the dataset relating to the patient discussed above in connection with step 210. Updating may include appending patient data based on the additional patient-related information received in step 325 to the dataset relating to the patient, or overwriting some or all of the existing content of the dataset relating to the patient with patient data that is based on the additional patient-related information received in step 325.

At this point, the process loops back to step 315. In this case the data processing device 105 processes the dataset, the patient position and the desired patient endpoint to generate the co-therapy regimen in the manner described earlier in connection with step 215.

The co-therapy regimen generated by this second iteration of step 315 can be the same or different to the co-therapy regimen generated by the first iteration of step 315. Any differences are attributable to data processing device 105 making use of a revised patient dataset that includes or is based on the additional patient-related information received in step 325.

Data processing device 105 makes use of the revised patient dataset to provide a recommended co-therapy regimen when performing the second iteration of step 315 that is responsive to the actual state of the patient. It will thus be appreciated that steps 315 to 330 can be repeated many times in the manner shown in FIG. 3 to enable a dynamic co-therapy regimen to be devised, which dynamic regimen is responsive to the actual state of the patient.

It will be appreciated that data processing device 105 may at any point receive a new desired patient endpoint, e.g. from patient device 115 or clinician data processing device 130. Responsive to receiving a new desired patient endpoint, data processing device 105 is configured to replace the existing desired patient endpoint with the new desired patient endpoint. Thus, at the next iteration of the process of FIG. 3, a co-therapy regimen is generated based on the new desired patient endpoint.

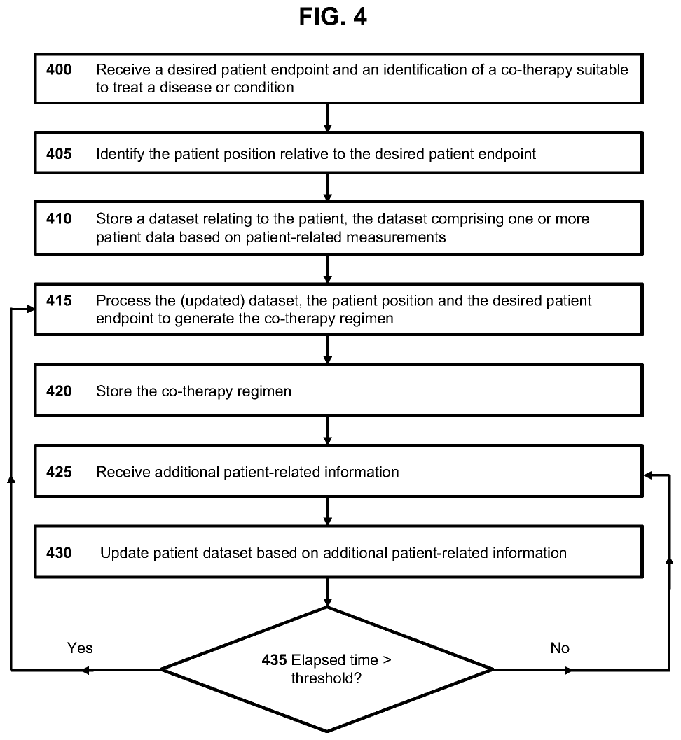

Another exemplary embodiment in which data processing device 105 performs more than one iteration is shown in FIG. 4. Steps 400 to 430 are the same as steps 300 to 330, respectively, so are not described in detail again here. The following sets out additional considerations that are preferably present in an iterative process like that of FIG. 4.

In step 435, data processing device 105 calculates whether an elapsed time associated with the additional patient-related information exceeds a threshold value. The elapsed time is equal to a length of time between the most recent update to the co-therapy regimen 415 and the most recent update to the patient dataset 420. A time at which a patient-related measurement was made can be established by generating a timestamp at the time the measurement was made, which timestamp can be appended to, or otherwise associated with, the measurement.

As an example, in the case where a processing device 105 generates an updated co-therapy regimen at 9 am and measures their blood sugar level at 12 pm on the same day, the elapsed time associated with the measurement is 3 hours.

The threshold value is set according to the considerations discussed earlier in this specification relating to the timeframe within which one would expect the patient to respond to the therapies, e.g. the time noted above between processing step (e) of two consecutive cycles in relation to the second aspect of the invention. As set out above, preferably the threshold value is set such that it is greater than or equal to the duration of a timeframe within which one would expect the patient to respond to the therapies and less than a time between two consecutive patient visits to a healthcare provider.

The threshold value may be fixed at the outset of a course of treatment and remain static throughout, or the threshold value may be varied as a course of treatment is ongoing, i.e. from iteration to iteration of the process of FIG. 4. Variations may be based on analysis of the timeframe over which a particular patient responds to a particular co-therapy, which analysis may be performed by data processing device 105 on the patient dataset established in step 310.

In the case that the elapsed time is calculated to be less than the threshold, the process returns to step 425 in which data processing device 105 awaits further patient-related information. Without being bound by theory, it is believed that it may be counterproductive in at least some cases to adjust the co-therapy regimen over a timeframe that is significantly shorter than an expected timeframe for the patient to respond to the therapies. An ‘overshoot/undershoot’ scenario where the regimen oscillates for some time before reaching a stable level may thus be avoided, or at least the time spent oscillating may be reduced.

In the case that the elapsed time is calculated to be greater than the threshold, the process loops back to step 415 and proceeds as described in respect of FIG. 3. It is preferred in such a case that any patient-related information gathered during one or more iterations in which the elapsed time was calculated to be less than the threshold time is used in the generation of the co-therapy regimen of step 415. In this manner, patient-related information that is gathered too rapidly for immediate processing is still made use of.

As in the case of the process of FIG. 3, it will be appreciated that data processing device 105 may at any point receive a new desired patient endpoint, e.g. from patient device 115 or clinician data processing device 130. Responsive to receiving a new desired patient endpoint, data processing device 105 is configured to replace the existing desired patient endpoint with the new desired patient endpoint. Thus, at the next iteration of the process of FIG. 4 for which the elapsed time is greater than the threshold, a co-therapy regimen is generated based on the new desired patient endpoint.

Thermodynamics dictates that energy is conserved. In terms of body weight, this means that overall energy input equals energy output. Energy input can be from new calories from food intake or from burning existing calories. Energy out is a combination of calories burned from basal metabolic rate together with calories burned through activity. If new calories from food intake are less than the calories burned, then the deficit is made up from burning up calories stored in the body, particularly from glycogen and fat stores.

The majority of energy is stored in fat, particularly in obese individuals. Therefore, to lose weight, fundamentally one needs to enter into a calorie deficit from the perspective of eating. i.e., one needs to be on a diet.

There are a profusion of diets with different food compositions and food timings including Atkins, 5:2 diet, Weight Watchers and the ketogenic diet. A problem with all diets is sustainability. Typically, an individual may manage to embark on a diet and see some early weight loss, but then the intrinsic drive from the parts of our brain controlling appetite, in particular the hypothalamus, drive behaviour to return to the previous weight set point through increased consumption of food. Indeed, the body will also try to maintain weight whilst in calorie restriction through a decrease in the basal metabolic rate. One long term consequence of the temporary shift in weight and then homeostatic drive to return to baseline can be an overshoot from prior baseline such that the individual eventually ends up at a higher weight.

Successful weight loss requires dietary restriction that can be sustained long term with an ultimate permanent lower calorie intake, ideally accompanied by increased physical activity (exercise) levels. Increasing the basal metabolic rate and maintaining the new lower weight set point can be facilitated by increased physical activity.

The drive to eat can be considered as reflecting two states—hunger and satiety. There is also a sense of fullness, but this can be dissociated from satiety. For example, a patient may feel bloated or have delayed emptying of food from their stomach yet still feel calorie depleted and hungry. The term satiety is used herein to define a patient's desire to eat and the term is intended to encompass a level of: hunger, fullness, satiety or any other similar measure of a patient's desire to eat.

The gut hormone GLP1 (glucagon like protein) facilitates weight loss by promoting a feeling of satiety (and fullness). It therefore makes it easier for the patient to calorie restrict. However, GLP1 will not achieve weight loss in the absence of calorie restriction.

The methods and systems of the present disclosure can treat obesity in a patient by administering a GLP1 agonist (which may be referred to herein as a GLP1) in a treatment regimen in conjunction with the patient engaging in a directed digital therapeutic program that manages the treatment regimen and patient behaviour to achieve a target weight for the patient. The disclosed methods and systems can advantageously coordinate the drug therapy and the behavioural therapy to achieve a target weight loss. The disclosed methods and systems can advantageously synchronise the timing of a behavioural regimen provided by an electronic device with the GLP1 treatment regimen, resulting in strong patient compliance with the treatment program and effective weight loss. As described below, the disclosed systems and methods can provide the treatment in a phased manner comprising a weight loss phase, a weight maintenance phase and a drug withdrawal phase. The treatment may incorporate a feedback loop which monitors patient progress data and adjusts a GLP1 dosage of the treatment regimen and/or the behavioural regimen to maintain patient compliance and stay on a patient weight loss trajectory towards a target weight with a minimum of side effects.

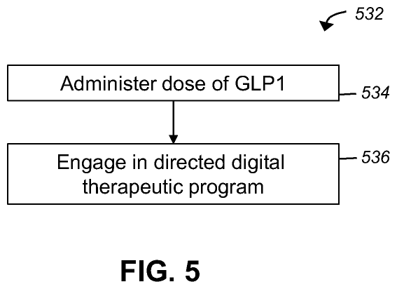

FIG. 5 illustrates a method 532 for treating obesity in a patient according to an embodiment of the present disclosure.

A first step 534 comprises administering a dose of a GLP1 agonist to the patient in a treatment regimen. The treatment regimen may be effective to manage the patient's satiety and thereby effect a reduction in the patient's weight.

A second step 536 comprises, in conjunction with the administration of the dose of GLP1 agonist of the first step 534, engaging in a directed digital therapeutic program that manages patient weight loss and the treatment regimen for the GLP1 agonist with the objective of achieving a predetermined target weight for the patient. The patient can achieve the target weight as a result of the first and second steps 534, 536.

In some examples, the digital therapeutic program (which may simply be referred to herein as the digital program) may provide a behavioural regimen to the patient. The behavioural regimen may comprise a calorie intake regimen and/or a physical activity regimen. The behavioural regimen may be delivered to the patient via an electronic device, for example the patient device, described above in relation to FIG. 1. The digital program may synchronise a timing of the behavioural regimen with a timing of the administration of the GLP1 dosage.

The digital program may also output instructions for delivering the treatment regimen. For example, the digital program may instruct the patient (or a clinician) to administer a dosage of the GLP1 agonist according to a dosage regimen. The digital program may provide the instruction via an electronic device. The digital program may output the dosage regimen as one or more dosage amounts and/or one or more corresponding dose timings to the patient.

The digital program may manage the patient weight loss and the treatment regimen in a four stage treatment protocol. The four stages may comprise: (i) an initial treatment titration phase; (ii) a progressive weight loss phase; (iii) a weight maintenance phase; and (iv) a drug withdrawal phase. In some examples, two or more of the stages may be combined. In some examples, stages (i) and (ii) may be grouped together and referred to as the weight loss phase. In some examples, stages (iii) and (iv) may be combined and referred to as the weight maintenance phase. In some examples, one or more stages may be separated into sub-stages.

By providing a staged approach, the treatment program can advantageously initiate and embed a behavioural change in the patient's lifestyle during the initial phases that is coordinated with the GLP1 treatment which suppresses their hunger. As the lifestyle changes become embedded, the patient can continue with a healthy diet and physical activity regimen as the GLP1 dosage is reduced or withdrawn during the drug withdrawal phase. In this way, the method can avoid chronic GLP1 dosing and patients can be weaned off the drug. This is particularly advantageous in jurisdictions where GLP1 treatment is only authorised for fixed durations (e.g. approved in the UK for 2 year treatment periods).

Initial Treatment Titration Phase

At the start of the treatment, the digital program may set an initial GLP1 dosage. As described herein, “setting or adjusting a GLP1 dosage” may refer to setting one or more dosage amounts or dosage timings of a dosage regimen of the GLP1 agonist. Setting or adjusting the GLP1 dosage may refer to setting or adjusting a dosage titration trajectory or ramp profile in which a dosage amount is gradually increased and/or decreased in discrete steps over a period of time (e.g. as the GLP1 is first introduced at the start of treatment or withdrawn at the end of treatment). The ramp up in the initial GLP1 dosage regimen may be scheduled as a series of stepwise escalations, for example increments in dosage every four weeks over a period of 12-24 weeks.

The digital program may set the initial GLP1 dosage based on a manufacturer's recommendation for the particular dosage form of the GLP1 agonist. In some examples, the digital program may set a personalised initial GLP1 dosage based on one or more of: patient data including patient genotype, patient phenotype, patient demographic data such as gender, ethnicity and/or age; patient physiological data such as patient weight, kidney function, basal metabolic rate, lean body mass and/or fat levels; and/or patient behavioural data such as calorie intake (including type of food consumed, water intake and timing (e.g. time since last meal), satiety, motivation to calorie restrict, and physical activity levels. The digital program may set the initial GLP1 dosage based on patient weight which is a key driver of GLP1 drug plasma level. The digital program may set the initial GLP1 dosage using a dosage calculator derived from a pharmacokinetic (PK) model. The digital program may set the initial dosage based on a target weight loss. For example, the digital program may determine a target plasma level to achieve the target weight loss based on a predetermined plasma level to weight loss relationship. The digital program may then determine the initial dosage based on the target plasma level and the patient initial weight. In some examples, the digital program may skip the intermediate calculation of the target plasma level and determine the initial dosage based on the target weight loss and the initial weight using a predetermined relationship such as a look up table. The predetermined relationship may be derived from patient study data and/or the PK model.

A patient phenotype, may provide an indication of the responsiveness of a patient to semaglutide or incretin drugs more generally. Examples of phenotypes include: (a)“hungry brain,” characterized by excessive calories consumed before eating is terminated; (b) “emotional hunger,” characterized by emotional eating and reward-seeking, in the face of otherwise normal homeostatic eating behaviours (39); (c) “hungry gut,” characterized by reduced duration of fullness, quantified objectively by rapid gastric emptying; and (d) “slow burn,” characterized by reduced REE, physical activity, exercise and muscle mass. People with a “hungry gut”, who tend to get hungry in between meals, have been shown to response best to GLP1 agonists. These patients may have low levels of naturally occurring GLP1 hormones which may be a cause of obesity and/or type 2 diabetes. As a result, such patients can have an above average response to GLP1 agonists.

The digital program may set an initial calorie intake regimen based on an initial weight of the patient and a target weight of the patient. The target weight may be agreed between the patient and a clinician when the treatment is prescribed. The clinician may also prescribe a treatment period over which the co-therapy will be administered to the patient and by which the target weight should be achieved. In some examples, the treatment period may be between 12 and 36 months, for example 24 months. In some examples, the treatment period may be indefinite. In some examples, the treatment period may only apply to the GLP1 regimen and the calorie intake regimen may be indefinite. The digital program may receive the initial weight and the target weight, for example via user input on a user interface of an electronic device. The digital program may set a weight loss trajectory based on the initial GLP1 dosage and/or the initial calorie intake regimen. The weight loss trajectory may be based on a typical weight loss versus dosage trend (exponential decay) from patient studies or from a PK model. The weight loss trajectory may also be based on an energy input and energy expenditure model. The weight loss trajectory may define a series of weight loss milestones between the initial weight and the target weight at periodic time points within the treatment period. The weight loss trajectory may include a prediction range, or expectation range, surrounding the nominal weight loss trajectory comprising an upper weight loss trajectory and a lower weight loss trajectory. The expectation range may define an acceptable weight loss trajectory.

The calorie intake regimen may include a calorie intake allowance, such as a calorie intake allowance for specific meals, a daily calorie intake allowance, a weekly calorie intake allowance etc. The calorie intake allowance may equate to a calorie restriction relative to the patient's calorie intake prior to the treatment. The digital program may determine the minimum degree of calorie restriction required to reach the target weight.

The digital program may receive dietary options data to indicate the range of dietary restrictions available to the patient, for example, whether or not a substitution diet is to available. The initial calorie intake regimen may provide the calorie intake regimen based on the dietary options data. For example, calorie intake regimen may instruct a substitution diet, if the dietary options data indicates one is available. While some patients may perform well with prescriptive meal substitution, others may only be able to access, or require, a more modest calorie restriction. For example, the initial calorie intake regimen may include recommendations to remove a single item from daily consumption, corresponding to the required calorie reduction as calculated to reach the desired weight. More generally, the calorie intake regimen may also instruct the consumption or avoidance of certain food types, e.g. whether protein, carbohydrate or fat, then subtype of food type, for example complex carbohydrate or simple sugar. The calorie intake regimen may also include instructions for a frequency of calorie consumption. For example, the calorie intake regimen may prescribe whether food should be eaten at regular time points during the day, whether meals should be skewed with a larger meal at one particular time, whether there are liquid calories and whether any snacks are permitted between meals.

The digital program may set a timing of the initial calorie intake regimen based on a start time of the GLP1 treatment regimen. For example, the treatment program may delay the onset of the calorie intake regimen by a drug effect time relating to the time for the GLP1 agonist to have an effect on the patient satiety (or a time for a drug plasma level to reach steady state). In this way, the digital program can coordinate the start of calorie restriction with the onset of the satiety effect of the GLP1. Delaying the initial calorie intake regimen can advantageously improve patient adherence (also referred to herein as concordance) to the calorie intake regimen as the patient perceives the new calorie regime as easier to achieve due to the hunger suppressing effect of the GLP1. As explained below, the digital program may modify the timing of the delay based on a motivation score of the patient. The intrinsic motivation to alter diet and calorie restrict can be highest at initiation of therapy, even though the actual effect on satiety and hunger will be less from the GLP1 as the dosage/plasma concentration has not yet increased to a steady state value and/or taken effect.

The digital program may receive other parameters for determining and setting the initial calorie intake regimen. For example, the digital program may receive a current calorie intake for the patient. The digital program may set a ramp profile of the initial calorie intake regimen to ramp from the current calorie intake to a reduced calorie intake allowance that can achieve the target weight loss. The digital program may also receive a motivation score of the patient indicating a motivation level of the patient for losing weight. The digital program may receive the motivation score as a scalar input on a user interface of the patient electronic device. The digital program may adjust the timing of the initial calorie intake regimen based on the motivation score. For example, the digital program may increase the ramp rate or reduce the drug effect delay time for patients with high motivation or vice versa.

The greater the calorie restriction, the greater the weight loss. However, a greater calorie restriction is behaviorally harder to achieve. Furthermore, as the patient loses weight, the homeostatic mechanism tending to revert weight to a particular set point becomes stronger and therefore it's harder to continue with the calorie restriction. Drug labels for GLP1s typically prescribe a ramp up in dosage amount over a number of weeks. The increasing dosage can address the increasing difficulty in adhering to a calorie restriction resulting from the homeostatic mechanism. However, the drug labels prescribe a fixed dosage ramp/titration trajectory and do not allow for individual variation in circumstances, motivation, drug efficacy, side-effects and other personalised influences on the treatment adherence and weight loss. The disclosed systems and methods can provide a personalised GLP1 dosage regimen in combination with a personalised behavioural regimen with both elements of the co-therapy advantageously adapting to the patient's personal treatment response as they progress through the co-therapy. In this way, greater patient adherence and sustained weight loss can be achieved even as the GLP1 is eventually withdrawn.

Following initiation of the GLP1 dosage regimen, the digital program can monitor patient progress data. The patient progress data may include one or more variables indicating the progress of their dietary restriction and weight loss. The patient progress data may include one or more of:
Patient weight loss data, for example regular (e.g. daily) patient weight measurements. The digital program may receive the patient weight loss data via manual user input from the patient or as a signal from electronic scales communicatively coupled with the electronic device. The weight loss data may include proportions of fat mass and lean mass. The digital program may calculate a moving average of patient weight due to the inherent fluctuation in weight, particularly when measured on scales owing to differences in food and fluid intakes and timing of bowel motions. The digital program may also incorporate a time lag before recommending a decision based on the patient weight data to avoid erroneous oscillation.
Patient calorie intake data. The patient may record their food intake. For example, the digital program may provide a database of foodstuffs and food types that the patient can select on the electronic device to indicate consumption. The calorie intake data may comprise a moving average calorie intake, for example an average daily calorie intake data over a period of 3 days or 7 days.
Patient motivation score. The digital program may receive a quantifiable input from the user to indicate their motivation to continue with the program and the calorie intake regimen. The digital program may provide a visual analogue scale or visual slider that the patient can manipulate on the patient electronic device. The digital program may receive the motivation score regularly, for example via daily prompts, and/or on demand. For example, the patient may activate a low motivation alert button when they experience intense cravings or low mood.
Patient satiety/hunger score. The digital program may receive a quantifiable input from the user to indicate a degree of satiety they are experiencing. The digital program may provide a visual analogue scale or visual slider that the patient can manipulate on the patient electronic device. The digital program may receive the satiety/hunger score regularly, for example via daily prompts or more regularly, and/or on demand. The digital program may monitor the patient satiety as a moving average to determine whether the patient is consistently experiencing hunger suppression. The patient satiety score may also encompass a patient fullness score and/or a patient hunger score. Satiety score may encompass a broader spread of measures. For example satiety score may include hunger (before meal), satiety (post meal), fullness (gastric emptying after meal may be fast or slow according to drug level). Satiety score may also encompass food preferences which can change in response to GLP1 therapy. However, food preferences may be captured as part of calorie intake data.
Patient side effect data. The digital program may receive reported side effect data via patient input. The digital program may prompt the patient with a list of potential side effects. The acquired data may include all GI disturbances such as nausea and vomiting. Potential side effects may include one or more of: nausea, diarrhea, vomiting, constipation, abdominal (stomach) pain, headache, fatigue, dyspepsia (indigestion), dizziness, abdominal distension, eructation (belching), hypoglycemia (low blood sugar) in patients with type 2 diabetes, flatulence (gas buildup), gastroenteritis (an intestinal infection) and gastroesophageal reflux disease (a type of digestive disorder). The patient may input side effects experienced and a severity level. The severity level may comprise a qualitative (low, medium, high) and/or quantifiable score (e.g. pain, nausea etc. on a scale of 1-10) which may be received via patient entry using a visual analogue scale or slider on the electronic device. The timing of nausea in relation to meals, duration and effect on function is also recorded. Furthermore, the effect of a side effect such as nausea (or any other side effect) on function may be recorded. A patient may have relatively mild nausea that they find markedly interferes with function, whereas another patient may note nausea as being quite severe, but are able to continue with function.
Patient activity data. The patient activity data may represent a level of physical activity undertaken by the patient and may include manual user entry or automated sensor entry using an activity sensor such as an accelerometer or motion sensor. Such sensors may be provided as part of the patient electronic device. The digital program may translate patient activity levels to ‘PAEE’ Physical Activity Energy Expenditure in Cals/day (or kJ/h etc): This might be a ‘conversion algorithm from steps entered by an individual or embedded electronically & associated with a wearable device.

The digital program may adjust the GLP1 dosage and/or the calorie intake regimen based on the patient progress data. Adjusting the GLP1 dosage may comprise increasing or decreasing a dosage amount of one or more scheduled dosages, adjusting one or more dosage timings of scheduled dosages, increasing or decreasing a ramp rate of a planned dosage increase or decrease etc. Adjusting the calorie intake regimen may comprise: increasing or decreasing a calorie intake allowance, increasing or decreasing a rate of change of the calorie intake allowance or adjusting instructions relating to timing of calorie intake or types of food consumed.

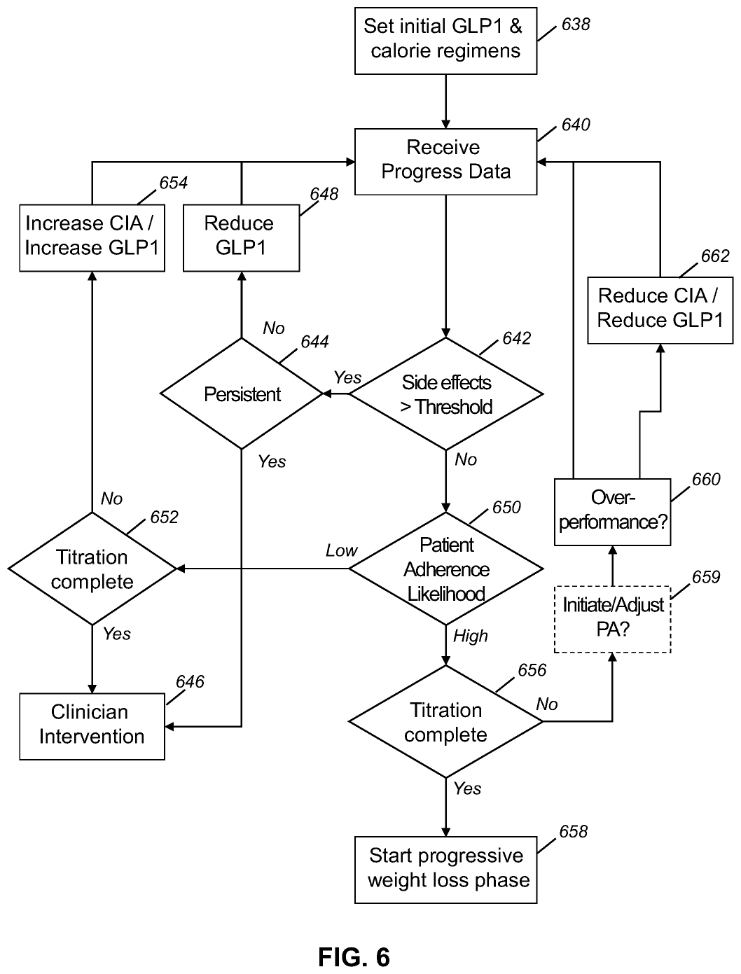

FIG. 6 illustrates a method of adjusting the treatment during the initial treatment titration phase according to an embodiment of the present disclosure. The method may be performed by the digital program.

A first step 638 comprises setting the initial GLP1 dosage and the initial calorie intake regimen as described above.

A second step 640 comprises receiving the patient progress data.

A third step 642 comprises determining if the patient side effect data is representative of a level of side effects greater than a side effect intolerance threshold. The side effect intolerance threshold may comprise a timeframe, for example a level of nausea has been unacceptable for a number of consecutive days. The intolerance threshold may simply comprise the patient reporting that they cannot tolerate one or more side effects.

If the level of side effects is greater than the intolerance threshold the method proceeds to decision point 644 to determine if the level of side effects have been persistently greater than the intolerance threshold, for example the intolerance threshold is persistently exceeded (over a number of iteration loops of FIG. 6) despite a reduction in GLP1 dosage or the patient is unable to tolerate a dosage level required for sufficient hunger suppression. If the side effects have been persistently greater than the intolerance threshold, the method proceeds to termination point 646 and clinician intervention. For example, the clinician may recommend an alternative treatment (e.g. surgery, different drugs). In this way, patients who experience persistent unacceptable side effects and are unsuitable for GLP1 therapy are rapidly identified. This can advantageously save cost for the patient and/or medical authority as GLP1 therapy can be expensive.

Returning to step 644, if the level of side effects being greater than the intolerance threshold is not persistent, the method may proceed to step 648 and reduce the GLP1 dosage. The GLP1 dosage reduction may be a reduction in dosage amount, a delay in a scheduled dosage or a reduction/increase of the rate of increasing/decreasing dosage amounts. In some examples, the reduction in GLP1 dosage may be a temporary—it has been shown that this improves over time when tolerance to the adverse side effects develop. In other examples, the reduction in GLP1 dosage may be permanent because the patient achieves greater weight loss and therapy concordance without the unwanted side effects. Following reduction of the GLP1 dosage the method returns to step 640 and receives further progress data to continue to monitor the side effects.

Returning to the third step 642, the step may also comprise monitoring the reported side effects data for signs of toxicity arising from the drug. The drug label for semaglutide includes warnings regarding the potential for thyroid cancer and endocrine neoplasia. The method may comprise proceeding to step 614 and alerting a HCP if the reported side effect data includes symptoms of toxicity.

Step 642 may also monitor the reported side effect data for a patient willingness to proceed despite reporting severe side effects. If the patient indicates a willingness to proceed the method may discount the sever side effects.

At step 642 If the level of side effects is less than the side effect intolerance threshold or the side effect data indicates a patient willingness to proceed, the method proceeds to step 650 and estimates the likelihood of patient adherence to the calorie intake regimen. The digital program may estimate the likelihood of patient adherence based on the patient progress data in a number of ways. For example, the digital program may predict a low likelihood of patient adherence to the calorie intake regimen if: (i) the patient weight loss data indicates that the patient weight loss is behind schedule, i.e. the patient weight loss is less than the acceptable patient weight loss trajectory; (ii) the patient calorie intake data represents a calorie intake greater than a first upper calorie intake threshold (e.g. a proportional amount, such as 25%, above the calorie intake allowance); (iii) the patient motivation score is less than a lower motivation score threshold (e.g. less than 4 on a scale of 0-10); or (iv) the patient satiety score is less than a first lower patient satiety threshold (e.g. less than 5 on a scale of 0-10). The threshold comparison conditions may include not meeting the threshold for a persistent time period e.g. a consecutive number of days or a proportion of consecutive days (e.g. exceeding the calorie intake allowance by more than 25% for three days out of four). Each of these conditions may indicate that the patient is having, or will have, difficulty adhering to the calorie intake regimen. The patient may experience such difficulty particularly during the initial stages of GLP1 treatment when the drug dosage has not reached a maximum dosage amount and/or has not taken full effect. Therefore, if the method determines that the patient adherence likelihood is low (e.g. by meeting one of the conditions (i)-(iv)) the method proceeds to step 652 to check if the initial GLP1 dosage titration is complete. The initial GLP1 dosage titration may be complete when the GLP1 dosage amount has increased to a maximum dosage amount and had sufficient time to have full effect in the patient (steady state drug plasma level).

If the initial GLP1 dosage titration is complete, the method proceeds to termination point 646 to seek clinician intervention because the maximum dosage and drug effect have failed to result in patient adherence to the calorie intake regimen. The clinician may provide better dietary options such as a substitution diet. For example, patients unable to maintain a 500 Cal/day reduction by reduced meal size/snacking may be prescribed an initial or even extended use of meal replacement options and return the patient to step 640. Alternatively, the clinician may stop the treatment and/or prescribe alternative or additional treatment (e.g. surgery or different drugs using a different mechanism of action such as metformin or topiramate). In some examples, the method may comprise adding such additional/alternative treatments to the co-therapy (either directly in response to step 652 or clinician adjustment) and adjust the dosages of the alternative/additional treatments in response to the patient data. In this way, patients who do not respond well to the therapy are rapidly identified which can advantageously save cost.

Returning to step 652, if the initial GLP1 dosage titration is not complete, the method proceeds to step 654 to adjust the GLP1 dosage and/or the calorie intake allowance to improve the patient adherence. For example, the method may temporarily increase the calorie intake allowance to allow the GLP1 dosage regimen to take effect in the patient. For example, the temporary increase may persist while the GLP1 dosage amounts are increased according to the initial GLP1 dosage titration trajectory resulting in associated improvements in the patient weight loss data, satiety score, motivation score, and/or calorie intake data. Alternatively, or in addition, the method may increase the GLP1 dosage. For example, the method may increase the ramp rate of the increasing dosage titration trajectory, bring forward a dosage increase or otherwise accelerate the GLP1 dosage increase. Following adjustment of the GLP1 dosage and/or calorie intake regimen, the method returns to step 640 and continues to monitor the patient progress data.

Returning to step 650, if the method determines that the patient adherence likelihood is high, the method proceeds to step 656 to check if the initial GLP1 dosage titration is complete, in the same way as described for step 652. The method may also check if the initial calorie intake regimen is complete (i.e. a maximum planned calorie restriction has been achieved). If the initial GLP1 dosage titration is complete, i.e. the maximum dosage has been reached and the progress data is indicating tolerable side effects and patient adherence, (and optionally the initial calorie intake regimen is complete) the method proceeds to step 658 to initiate the second phase of the protocol—the progressive weight loss phase.

Returning to step 656, if the initial GLP1 dosage titration is not yet complete, this indicates that a GLP1 dosage ramp-up and/or a calorie intake allowance ramp down is ongoing. In other words, the patient is on track. The method can proceed to optional step 659 and determine whether a physical activity (PA) regimen should be initiated or adjusted. The method may recommend timing of, initiation of, duration and intensity of physical activity (e.g. specific exercise) to correspond with the capabilities of the patient at a particular stage of their treatment process. The timing and intensity of the physical regimen may be titrated based on measurement of motivation and achievability as indicated by the progress data. The method may introduce a physical activity regimen if the weight loss data indicates patient weight loss exceeding a first weight loss milestone. Increased physical activity such as exercise can be easier to initiate when a patient has already undergone some weight loss. A small amount of weight loss can lead to a significant change in pressure on the patient's joints and cardiovascular system, thereby allowing increased activity. Furthermore, administering GLP1 and restricting calories will lead to a loss of both fat mass and lean mass in an approximately 3 to 1 ratio. A physical activity regimen can protect against the loss of lean mass. The method may increase a level of physical activity intensity if: the weight loss data indicates patient weight loss exceeding one or more further weight loss milestones; the patient motivation score exceeds a second motivation threshold; or the patient activity data satisfies one or more activity thresholds. It will be appreciated that the method may also decrease an intensity of the physical activity regimen, for example at step 654 if the patient has low motivation. In some examples, the method may only introduce a physical activity regimen in later phases, such as the progressive weight loss phase or the weight maintenance phase.

Following step 659, the method proceeds to step 660 and assesses whether the patient adherence or weight loss is exceeding expectations. The method may determine the patient adherence or weight loss to be exceeding expectations if: (i) the patient weight loss data represents a weight loss ahead of schedule, i.e. exceeding the acceptable weight loss trajectory; (ii) the patient calorie intake data represents a calorie intake less than a first lower calorie intake threshold (e.g. a proportional amount, such as 25%, below the calorie intake allowance); (iii) the patient motivation score is greater than an upper motivation score threshold (e.g. greater than 7 on a scale of 0-10); or (iv) the patient satiety score is greater than a first upper patient satiety threshold (e.g. greater than 7 on a scale of 0-10). The threshold comparison conditions may include exceeding the relevant threshold for a persistent time period e.g. a consecutive number of days or a proportion of consecutive days (e.g. consuming less than the calorie intake allowance by more than 25% for three days out of four). Each of these conditions may indicate that the patient is exceeding the weight loss and/or adherence expectations.

If the method determines that the patient adherence or weight loss is exceeding expectations, the method proceeds to step 662 and adjusts the GLP1 dosage and/or the calorie intake regimen accordingly. For example, the method may reverse an increase in calorie intake allowance that was applied at step 654 at an earlier stage of the treatment to address poor adherence. Alternatively, patients may be able to adopt diets with greater energy restriction, and optionally increased energy expenditure via physical activity, while remaining on a relatively low doses of GLP1. Alternatively, or in addition, the method may reduce the GLP1 dosage. For example, the method may reduce, delay or cancel future dosage increases of the GLP1 titration trajectory. In this way, the method may complete the initial GLP1 dosage titration ahead of schedule, such that the method will proceed to step 658 on the next iteration. In some examples, the method may reduce the target weight to a more ambitious target, optionally seeking clinician input for approval of the new target weight. Such an approach may be accompanies by a further reduction in calorie intake allowance. Patients may engage strongly with the formal energy intake restriction and increased physical activity elements of the protocol. Such patients may realise sufficient personal reserve to tolerate a more rapid weight loss programme arising from greater restriction of energy intake. Following step 662, the method returns to step 640 and continues to monitor the patient progress.

If the method determines that the patient adherence or weight loss is not exceeding expectations, the method may maintain the current GLP1 dosage and calorie intake regimen and return to step 640 to await further patient progress data.

The method of FIG. 6 may be performed iteratively and periodically, for example daily, weekly etc. Step 640 may be performed more frequently, e.g. satiety data and calorie intake data may be received multiple times per day. Such data may be stored and assessed on a moving average basis. The assessment of such data, particularly at steps 642, 650 and 658 and any corresponding adjustments at 648, 654 and 662 may be performed with a periodicity that allows time for any GLP1 dosage adjustments to take effect. In some examples, the iterations may correspond to the periodic timing of the weight loss milestones or scheduled reviews. In this way, the burden of the digital component of the therapy can be kept to a minimum for patients who are not technologically literate.

The method of FIG. 6 can initialise and provide a personalised GLP1, calorie intake and physical activity co-therapy regimen optimised for side effect mitigation and achievement of satiety. The stringency of calorie restriction can be adjusted according to the moving average of satiety or a related measure. If there is a desire for greater than the calorie intake allowance then a lower level of calorie restriction is recommended until a higher GLP1 dosage is reached and the moving average of satiety improves sufficiently to enable the greater degree of calorie restriction (unless the patient has high levels of motivation to temporarily live with uncomfortable degrees of hunger until the new GLP1 dose is reached). The method of FIG. 6 can improve adherence rates and the effectiveness of GLP1 weight loss therapy because the method advantageously coordinates the timing of the calorie intake restriction with the effects of the GLP1. In this way, the method can reduce premature drop-outs from GLP1 therapy. However, the method can also rapidly identify patients who are not suitable for GLP1 therapy due to poor response and/or intolerable side effects.

The initial treatment titration phase can initiate the co-therapy in a personalised manner and adjust the co-therapy in a personalised manner in response to the patient progress data. In this way, the method can initiate each patient in a personalised way and advance them on to the progressive weight loss phase when the patient: has reached a stable GLP1 dosage delivering a stable satiety level; is adhering to a restricted calorie allowance to promote weight loss; and is optionally engaging in a complementary physical activity regimen.

Progressive Weight Loss Phase

The protocol may proceed to the second phase—progressive weight loss phase following completion of the initial GLP1 dosage titration trajectory and optionally completion of the initial calorie intake regimen (completion of a calorie restriction ramp). The goal of the second phase is to reach a stable GLP1 dosage that provides persistent weight loss towards the target weight over a period of several months. The method may also comprise determining and maintaining a minimum dosage of GLP1 that will provide a patient satiety sufficient for the patient to adhere to the calorie intake regimen and the acceptable weight loss trajectory. GLP1s can be expensive, so maintaining the minimum sufficient GLP1 dosage can advantageously save cost for the patient and/or healthcare system.

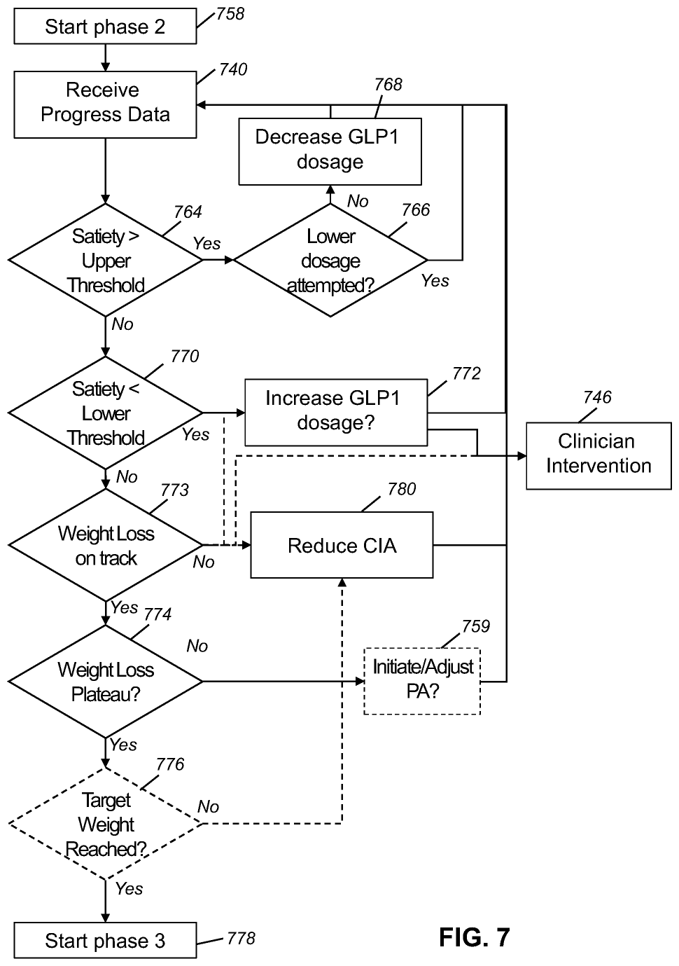

FIG. 7 illustrates a method of achieving a stable co-therapy regimen for the progressive weight loss phase according to an embodiment of the present disclosure.

The method picks up where FIG. 6 left off at step 758 and commences the progressive weight loss phase. In the same way as described above for FIG. 6, the method iteratively and periodically receives 740 patient progress data. The period of the iteration may be the same as the first phase or may be longer because the GLP1 and calorie intake regimens are more stable. In the progressive weight loss phase, patient weight loss data and satiety score are two of the key metrics monitored, however the other progress data parameters (e.g. motivation, side effects etc) may be continued to be monitored, with the method responding accordingly.

As noted above, the second phase may determine a GLP1 dosage that controls hunger sufficiently to achieve consistent weight loss towards the target weight. The second phase may iteratively adjust the GLP1 dosage up or down in order to maintain a level of satiety that continues to promote weight loss. There may be a number of ways of monitoring whether satiety is sufficient to promote weight loss. For example, simply ensuring that satiety remains higher than a threshold and increasing the GLP1 dosage amount if not. The method of FIG. 7 also checks if satiety is sufficiently high to suggest that the GLP1 dosage may be reduced. It may be advantageous to maintain as low a GLP1 dosage as possible to reduce cost, improve the prospects for successful drug withdrawal and to minimise side effects. Although FIG. 7 focusses on satiety, other example methods may additionally or alternatively use other parameters such as calorie intake data to assess whether the patient's hunger is sufficiently controlled.

The method proceeds to step 764 to check if the patient satiety score is greater than a second upper patient satiety threshold (e.g. satiety score of 8 on a scale of 0-10). The second upper patient satiety threshold may be set at a level that indicates that a GLP1 reduction could be implemented and maintain a satiety level sufficient for weight loss. The second upper patient satiety threshold may be the same as the first upper patient satiety threshold. If the patient satiety score is greater than the second upper satiety threshold, the patient may be able to tolerate a lower dosage and still experience a satiety score sufficient for weight loss. Therefore, the method proceeds to step 766 to check if a lower GLP1 has previously been attempted (and unsuccessful). If so, the method maintains the current GLP1 dosage and returns to step 740. If the lower GLP1 dosage has not been previously attempted, the method proceeds to step 768 and reduces the GLP1 dosage before returning to step 740. In this way, the loop 740, 764, 766, 768 can ensure the GLP1 dosage is at a minimum level sufficient to promote weight loss towards the target weight. In some examples, an alternative to the steps of 766 and 768 may be to reduce the target weight for the patient to a more ambitious target, optionally including seeking clinician approval. Such an approach may be accompanied by a further reduction in the calorie intake allowance (which could take effect at step 780 described below).

Returning to step 764, if the satiety score is not greater than the second upper patient satiety threshold, the method proceeds to step 770 to check if the satiety score is less than a second lower patient satiety threshold. The second lower patient satiety threshold may indicate that the patient is at risk of not complying with the calorie intake regimen or not losing weight. The second lower patient satiety threshold may be the same as the first lower patient satiety threshold. Immediately following the first phase of FIG. 6, the satiety score should be greater than the first lower satiety threshold because it was a condition of exiting the first phase at step 650. However, on later iterations, patient satiety may increase following a reduction in GLP1 dosage (e.g. at step 768), a reduction in calorie intake allowance (step 780) and/or a change in personal circumstances, such as life events (stress, temporary illness, loss of a loved one etc). If the satiety threshold is less than the second lower satiety threshold, the method proceeds to step 772 and attempts to increase the GLP1 dosage. The increase in GLP1 dosage may be dictated by whether the patient is currently on a maximum allowable dosage. If the patient is currently on the maximum allowable dosage, the dosage may be maintained and/or clinician intervention 746 may be sought. The clinician may stop treatment, recommend complementary treatment or adjust the target weight (see below). Following step 772 the method returns to step 740.

The combination of steps 764 and 770 provide a check that the satiety score is within an optimal range—higher than the second lower satiety threshold to promote sufficient weight loss and lower than the second upper satiety threshold which is indicative of an excessive GLP1 dosage. Step 766 prevents yo-yoing between dosage levels either side of a sufficient satiety level by biasing the patient towards a higher dosage level.

The use of two separate thresholds to define a satiety threshold range also reduces the risk of yo-yoing dosages.

Returning to step 770, if the patient satiety score is not less than the second lower satiety threshold, the method proceeds to step 773 to check if the weight loss is on track, for example if the weight loss is within the acceptable weight loss trajectory. If the weight loss is less than the acceptable weight loss trajectory, in other words the patient is not losing weight fast enough, the method may also, as part of step 773, determine if the calorie intake data represents a calorie intake less than the calorie intake allowance to ensure the patient is complying with the calorie intake regimen. The method may also comprise checking activity data to determine if the patient is adhering to a physical activity regimen. If the patient is complying with the calorie intake regimen (and optionally the physical activity regimen), the method may proceed to step 780 and reduce the calorie intake allowance before returning to step 740. If the patient is not complying with the calorie intake regimen (and optionally the physical activity regimen), the method may proceed to step 772 to increase the GLP1 dosage or to step 746 and seek clinician intervention to address the compliance issues. Returning to step 773, if the weight loss exceeds the weight loss trajectory, the method may jump to step 766 described above or continue on to step 774 described below.

If the weight loss is determined to be on track, or optionally exceeding the acceptable weight loss trajectory, the method proceeds to step 774 to check if the weight loss has plateaued, which is an indication that the third phase of the protocol should commence. The method may check if the patient weight loss data indicates that a rate of weight loss has been below a threshold weight loss rate for a period of time exceeding a stability time threshold. The stability time threshold may comprise a plurality of weeks such as 4 weeks, 8 weeks or 13 weeks.

If the weight loss has not plateaued, indicating the patient is on track and progressing through the progressive weight loss phase, the method may proceed to optional step 759 and initiate or adjust a physical activity regimen. This step may proceed in the same way as described above for identical step 659 of FIG. 6. As the weight loss phase progresses, the method advantageously begins to embed a new lifestyle of reduced calorie intake and regular exercise that will support the patient when the GLP1 therapy is reduced or withdrawn. Following step 759 the method proceeds to step 740 for further iteration. The progressive weight loss phase may also introduce, or increase a level of, a physical activity regimen at other points in the method of FIG. 7 (not illustrated). The physical activity regimen may be utilised as an extra lever to encourage compliance and or weight loss. For example, if the method determines that the weight loss is less than the acceptable weight loss trajectory at step 773, the method may introduce or increase an intensity of the physical activity regimen in response before returning to step 740. Increasing physical activity can improve patient mood and/or increase the basal metabolic rate thereby increasing the number of calories burned.

If the weight loss has plateaued, the method may proceed to optional step 776 and check if the target weight has been reached (within an acceptance threshold). If the target weight has been achieved the method may proceed to step 778 and start the weight maintenance phase. If the target weight has not been achieved, the method may proceed to step 780 and reduce the calorie intake allowance before returning to step 740 for a further iteration. As weight is lost, as well as an increase in homeostatic drive to revert to the previous weight, or an intermediary weight, the basal metabolic rate may also decrease, meaning the total calorie requirement for steady state becomes lower than baseline. Therefore, if further weight loss is desired, an additional calorie restriction can be provided at step 780 to provide sufficient hunger suppression. On subsequent iterations, the method may increase the GLP1 dosage at step 772 to address any drop in satiety resulting from the increased calorie restriction from step 780. In this way, the further calorie restriction may be accompanied with an increase in GLP1 dosage. However, the method may determine that the patient is unable to adhere to the new calorie intake allowance, for example by reviewing the patient progress data or because they are already on the maximum GLP1 dosage. In such circumstance, the method may increase the target weight such that step 776 is satisfied and the patient can commence the weight maintenance phase. In some examples, a clinician may adjust the target weight at step 746.

Returning to step 774, in some examples, if the weight has plateaued, the method may proceed straight to step 778, regardless of whether the patient weight has fallen to the target weight. In this way, the method reaches a realistic sustainable weight loss for the patient before commencing the maintenance and withdrawal phases.

Although not illustrated in FIG. 7, the method may continue to monitor side effects during the progressive weight loss phase, for example in the same way as described above in relation to FIG. 6. This may be particularly important following a GLP1 dosage increase at step 772.

The progressive weight loss phase of FIG. 7 focusses on monitoring: (i) patient weight loss to ensure weight loss is on track towards the target weight; and (ii) the patient satiety score to ensure that the patient adherence to the restricted calorie allowance is maintained. The method continues to embed a health lifestyle/behaviour in the patient including a restricted (and healthy) calorie intake and a physical activity regimen. Once a plateaued weight is achieved, ideally at the target weight, the method may proceed to the weight maintenance phase.

Weight Maintenance Phase

As weight loss approaches plateau, typically occurring around week 44 to 46 since treatment onset, phase three is entered in which calorie levels are set to maintain a steady state of weight, facilitated by the lowest GLP1 dosage that sufficiently controls hunger.

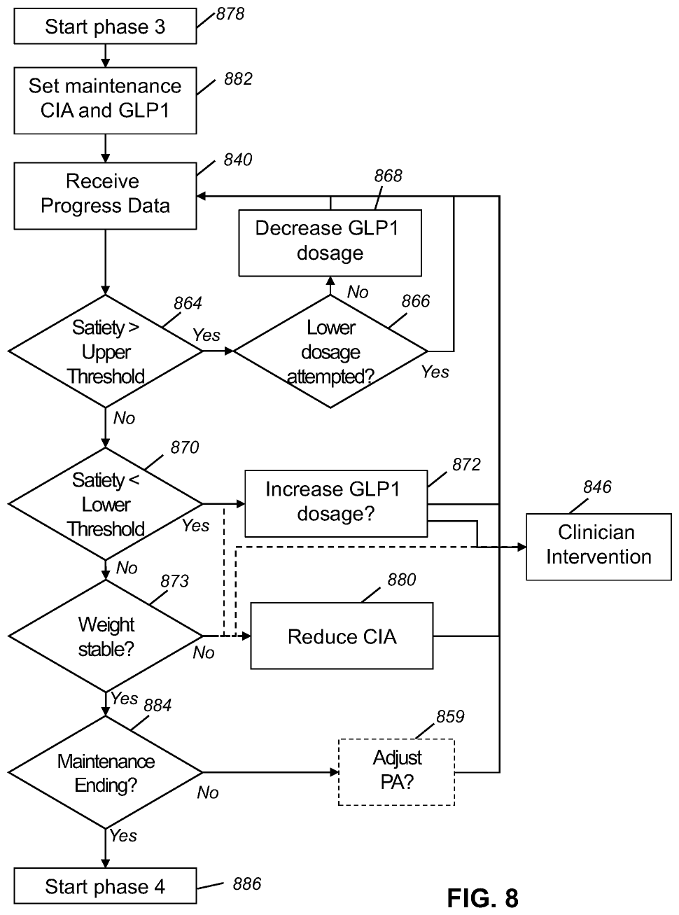

FIG. 8 illustrates a method of achieving a stable co-therapy regimen for the weight maintenance phase according to an embodiment of the present disclosure. The method has many identical steps to the method of the progressive weight loss phase 30 described in relation to FIG. 7. Identical steps have been given the same numbering in the 800 series and may not be explicitly described again here.

Following the start of the weight maintenance phase at step 878, the method proceeds to step 882 to set a maintenance calorie intake regimen. As the patient is moving from a phase of weight loss to a phase of weight maintenance, the calorie intake allowance may be increased. The method may also set a reduced GLP1 dosage to accompany the increased calorie intake allowance because the increased calorie intake will naturally improve the patient satiety score.

At this stage, the method may also set a maintenance weight, which may be the same as, or a proportion higher, than the target weight or the weight achieved at plateau.

The method proceeds to step 840 to receive progress data on an iterative and periodic basis. The period of the iteration may be the same as the first phase and/or the second phase or may be longer because the GLP1 and calorie intake regimens are more stable. Steps 864 to 872 operate in the same way as described above in that the patient satiety score is maintained within an optimal range at the minimum GLP1 dosage that provides hunger suppression sufficient for weight maintenance.

Picking up at step 873, if the patient satiety score is not less than the second lower satiety threshold, the method proceeds to step 873 to check if the patient weight is stable. For example, the method may check that the patient weight is within a threshold range, such a threshold range around the target weight. If the patient weight increasing outside the threshold range, in other words the patient weight has started to increase again, the method may also, as part of step 873, determine if the calorie intake data represents a calorie intake less than or equal to the calorie intake allowance to ensure the patient is complying with the calorie intake regimen. The method may also comprise checking activity data to determine if the patient is adhering to a physical activity regimen. If the patient is complying with the calorie intake regimen (and optionally the physical activity regimen), the method may proceed to step 880 and reduce the calorie intake allowance before returning to step 840. If the patient is not complying with the calorie intake regimen (and optionally the physical activity regimen), the method may proceed to step 872 to increase the GLP1 dosage and improve patient satiety or proceed to step 846 and seek clinician intervention to address the compliance issues. Returning to step 873, if the patient weight is within the threshold range, the method may proceed to step 884.

At step 884, the method determines if the patient is at the end of the weight maintenance phase. The method may determine this in a number of ways. For example, the method may determine that the patient is at the end of the weight maintenance phase if a remaining time of the treatment period is less than a withdrawal phase minimum duration. For example, the withdrawal phase minimum duration may comprise a time between two months and 12 months that is set aside for weaning the patient off the GLP1 therapy or down to a chronic maintenance dose. The method may also determine that the patient is at the end of the weight loss maintenance period if a GLP1 dosage has decreased from the GLP1 dosage set in step 882 by a threshold amount. If the patient is at the end of the maintenance period, the method proceeds to step 886 to start the drug withdrawal phase. Otherwise the method proceeds to optional step 859 before returning to step 408 for further iterations. Optional step 427 may operate in the same way as described above for steps 227 and 327 of FIGS. 2 and 3. The physical activity regimen in the maintenance phase may include strength or muscle building routines to rebalance fat mass and lean mass to protect against the loss of lean mass during the weight loss treatment. Altering the ratio of fat mass and lean mass to favour lean mass can also raise the Basal metabolic rate (BMR), slowing weight regain and improving the prospects for successful drug withdrawal, and permanent weight loss and lifestyle change. The weight maintenance phase may also introduce, or increase an intensity level of, a physical activity regimen at other points in the method of FIG. 8 (not illustrated). The physical activity regimen may be utilised as an extra lever to encourage compliance and or weight loss. For example, if the method determines that the patient weight loss has started to creep up again at step 873, the method may introduce or increase an intensity of the physical activity regimen in response before returning to step 840. Increasing physical activity can improve patient mood and/or increase the basal metabolic rate thereby increasing the number of calories burned.

Although not illustrated in FIG. 8, the method may continue to monitor side effects during the weight maintenance phase, for example in the same way as described above in relation to FIG. 6. This may be particularly important following a GLP1 dosage increase at step 872.

The weight maintenance phase of FIG. 8 establishes a patient stable weight while continuing to embed a healthier lifestyle in the form of a reduced calorie intake and regular physical activity. By the end of the maintenance phase, the method can establish the healthier lifestyle as a patient habit. Eating patterns can be both environment and habit dependent. Therefore, if there is a successful period of enacting a new eating pattern, this may become easier to sustain, independent of any effect of the GLP1 or intrinsic dietary change motivation. Furthermore, the obesogenic environment may have also changed as a result, for example, with different foods now filling the cupboards, skills acquired in preparing different sorts of meals and a different structure to daily activities including the physical activity regimen. Establishing the healthier lifestyle as a patient habit can provide optimal preparation for the withdrawal or reduction in the GLP1 dosage in the drug withdrawal phase. Such habit forming can be supported by the method providing education and lifestyle modules as described below.

Drug Withdrawal Phase

During the drug withdrawal phase, the GLP1 is gradually withdrawn, monitoring satiety levels and thus risk of relapse. Weight increase or satiety decrease and calorie rise can trigger either a short course of minimal dose GLP1 to re-establish desired behaviours, or a chronic maintenance dose.

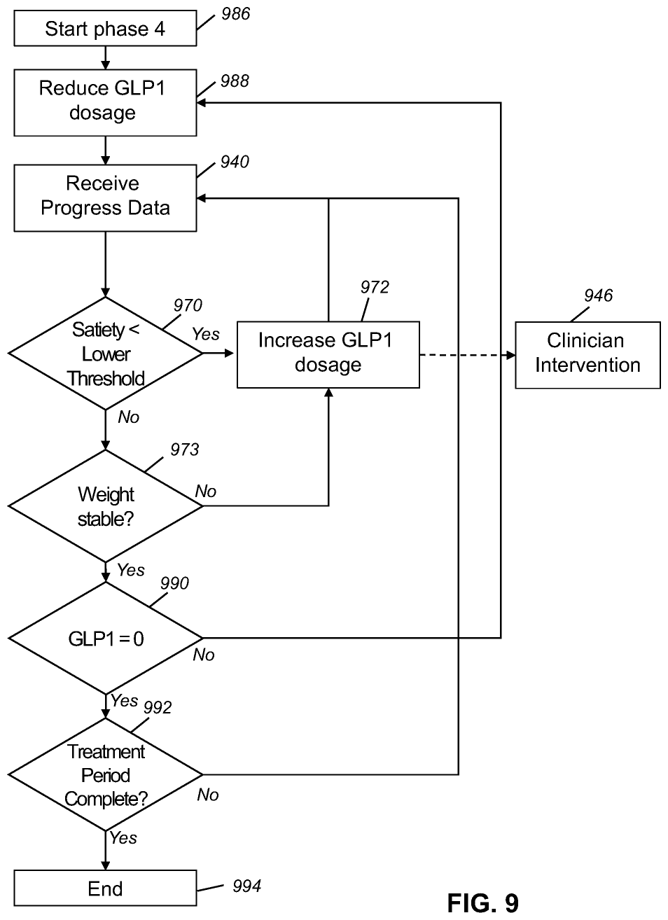

FIG. 9 illustrates a method of withdrawing GLP1 from a co-therapy regimen for the drug withdrawal phase according to an embodiment of the present disclosure.

Following the start of the drug withdrawal phase at step 986, the method proceeds to step 988 and decreases the GLP1 dosage. Decreasing the GLP1 dosage may comprise reducing a dosage amount or reducing a dosage frequency of the GLP1. The method proceeds to step 940 to receive patient progress data, in the same way as described above for the other phases.

At step 970, the method determines if the patient satiety score is less than a third lower satiety threshold. The third lower satiety threshold may indicate a risk of patient relapse to gaining weight and exceeding the calorie allowance. The third lower satiety threshold may be the same as the first and/or second lower satiety threshold. If the patient satiety score is less than the third lower satiety threshold the method proceeds to step 972 and increases the GLP1 dosage. The increase may be an increment back to the most recent higher dosage or, if the low satiety persistently returns every time the dosage is lowered towards zero, to a chronic maintenance GLP1 dosage. The method may optionally seek clinician intervention at step 946. The clinician may recommend complementary therapy such as surgery, or additional drugs using a different mechanism of action such as Phentermine/topiramate. The clinician may also recommend that the patient is transferred back to any of phases 1 to 3.

Returning to step 970, if the patient satiety score is greater than the third lower satiety threshold, the method may proceed to step 973 to check if the patient weight is stable. For example, the method may check that the patient weight is within a threshold range, such as a threshold range around the target weight. If the patient weight is increasing outside the threshold range, in other words the patient weight has started to increase again, the method may proceed to step 972 to increase the GLP1 dosage and improve patient satiety. Returning to step 973, if the patient weight is within the threshold range, the method may proceed to step 990.

At step 990, the method checks if the current GLP1 dosage is zero. If not, the method returns to step 988 and reduces the dosage further. In some examples, step 990 may also include checking if a time since the last dosage change is greater than a drug effect time. If the time is greater, the method may proceed to step 988, otherwise the method may return to step 940. In this way, the method avoids reducing the drug dosage too rapidly before the patient experiences the resulting reduction in satiety effect. If the GLP1 dosage is already zero, the method proceeds to step 992 to check if the treatment period is complete. If so, the method ends at step 994, If not the method returns to step 940 for further iteration, optionally via a physical activity regimen adjustment (not shown) similar to that described at steps 659, 759 and 859.

In some jurisdictions, GLP1 may be prescribed with a maximum treatment period, for example two years. In other jurisdictions, such as the USA, there is no restriction, however, having a finite treatment period can still save cost and end any side effects. The drug withdrawal phase can be particularly advantageous for patients who achieve weight targets within the 44-60 weeks as the remainder of the two-year approved prescription period can be used for the drug withdrawal phase. The drug withdrawal phase can flexibly assist with weight loss maintenance transition, such as a slow, step-wise reduction in dose or for relapse prevention, re-escalating the GLP1 dosage to catch and attenuate weight gain while further lifestyle support can be provided to assist the patient with the transition back to weight maintenance.

In some examples, the weight maintenance phase (FIG. 8) and the drug withdrawal phase (FIG. 9) may be combined into a single weight maintenance phase. For example, the outer loop 988 and 990 of FIG. 9 can be introduced into FIGS. 8 to controllably withdraw the drug. In such examples, a further phase may monitor the patient after drug withdrawal and indicate episodic bursts or a chronic prescription of GLP1 treatment and/or lifestyle education to address any relapse/weight gain above the target weight.

Lifestyle Education

Successful weight maintenance can be extremely challenging for the patient and suitable clinical service resources may often not be available or readily accessible. Research has shown that successful weight maintenance requires the individual to be equipped with knowledge, strategies, techniques and resources in advance of the weight maintenance programme. The disclosed method may incorporate education and training for patients to manage their behavioural change throughout the treatment process and the transitions between phases. In particular, the method may deliver lifestyle education via the patient electronic device, in the form of videos, slideshows, podcasts, forum and buddy support, interactive modules etc. The education modules may provide one or more of: dietary education, including food theory, cooking skills; physical activity/exercise education; mindfulness education for improving mood, improving motivation, reducing stress, reducing anxiety etc; education on improving the patients environment to promote healthy lifestyle and reduce calorie consumption triggers; and education on the treatment itself, such as an explanation of how the treatment will evolve, what to expect and how to address side effects.

The disclosed methods may introduce these education and lifestyle modules at any point during the treatment program. For example, the method may provide one or more education and lifestyle modules during the initial treatment titration phase when patient motivation for change may be high. The method may also provide one or more modules in accordance with an associated therapy change, for example provide the physical activity education at or prior to any of steps 659, 759 or 859 of the respective FIGS. 6 to 8. The method may also provide one or more modules in response to the assessment of patient progress data. For example: side effect education may provided in concordance with steps 642 and 644; dietary education may be provided with steps 650, 654, 662, 770, 880, 972; and mindfulness education may be provided in response to a low motivation score indicated by the patient progress data at steps 640, 740, 840 and 940.

As one example, a side effect education module may reassure a patient about the potential for waning side effects. Patient studies indicate that reported nausea drops with increased duration of any particular dose when maintained at that level. This adaptation may result from changes in patient physiology, but may also result from behavioural adaptations that the patient may undertake, such as eating smaller meals with, if necessary, increased frequency. The method may provide the side effect education module for educating the patient on such tolerance build up and/or providing advice to reduce the effects of nausea, prior to indicating a dosage reduction at step 648.

The method may provide a plurality of lifestyle modules prior to the drug withdrawal phase to embed the behavioural and lifestyle change in the patient that will complement the calorie reduction and physical activity regimen and maximise the prospects for successful drug withdrawal.

The success of weight loss and weight maintenance may also be strongly influenced by behaviours of individuals close to the patient and the food environment within which they live. Although not shown, the method may engage with other individuals of importance to achieving weight loss or maintaining weight loss, such as equipping such individuals to support the patient via education, strategies, techniques, behaviours and environmental change.

Approval Mechanisms

Throughout the described methods, any recommended adjustment to the co-therapy, particularly recommended changes to the GLP1 dosage may be subject to patient and/or HCP approval. For example, the method may comprise seeking patient approval for a recommended GLP1 dosage change. For example, a prompt may be provided on the patient electronic device. If the patient does not indicate a willingness to proceed, the method may comprise maintaining the current GLP1 dosage. If the patient indicates a willingness to proceed, the method may proceed to indicate, and/or instruct administration of, the recommended dosage change.

Other Example Implementations

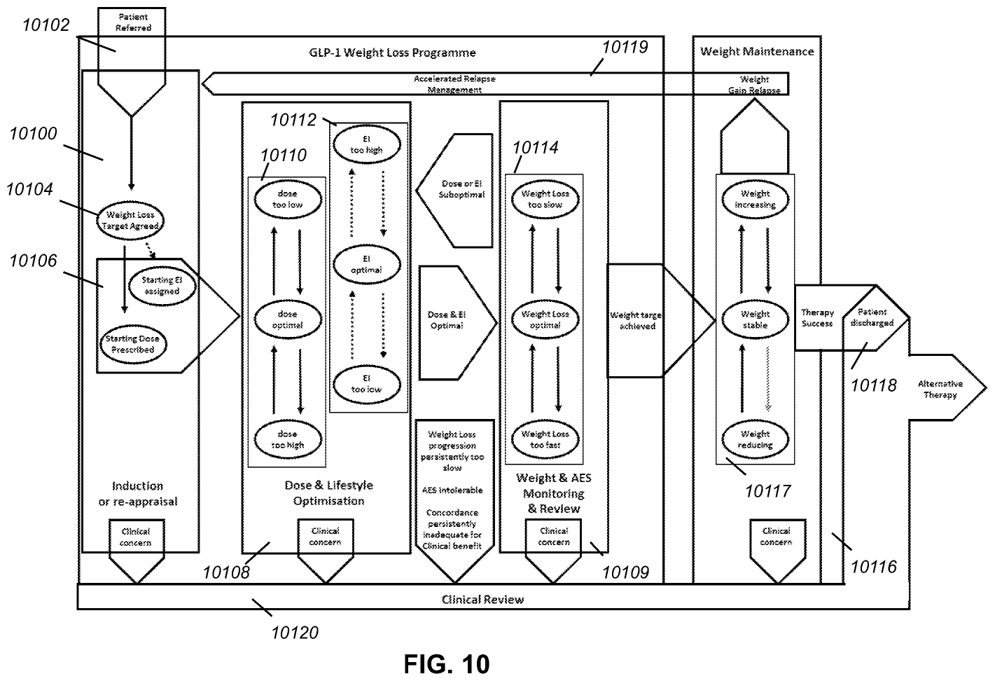

FIG. 10 illustrates a state diagram for the GLP1 co-therapy according to an embodiment of the present disclosure.

In an initialisation phase 10100, after referral 10102, the patient and HCP agree a target weight 10104. The method proceeds to step 10106 and sets an initial GLP1 dosage and an initial calorie intake regimen (labelled EI—energy intake) as described above in relation to FIG. 6.

After setting the initial regimens, the method proceeds to a weight loss phase comprising a treatment titration routine 10108 and a progress monitoring routine 10109. The weight loss phase may comprise the Initial Treatment Titration Phase and the progressive weight loss phases described above. During the_weight loss phase, the method reviews patient progress data including weight loss data 10114 and side effect data (not shown) in the progress monitoring routine 10109 and updates 10110 the GLP1 dosage (up, down, maintain) and updates 10112 the calorie intake regimen (up, down maintain), if the weight loss or reported side effects are suboptimal.

Following the weight loss phase, the method proceeds to a weight maintenance phase 10116 if the target weight has been achieved. The method continues to monitor 10117 patient weight during the weight maintenance phase and if the weight remains optimal, the patient is discharged 101118 after expiry of the treatment duration. If the patient weight is unstable, for example the weight increases, the method may return 10119 the patient to an earlier phase.

Throughout the process, the patient is monitored for HCP referral. For example, if weight loss progression is too slow, side effects are intolerable or patient concordance with the therapy (particularly the calorie intake regimen) is persistently inadequate, the method may refer the patient for clinical review 10120. HCP input may also be sought to review progress, for example at the periodic time points corresponding to the weight loss milestones.

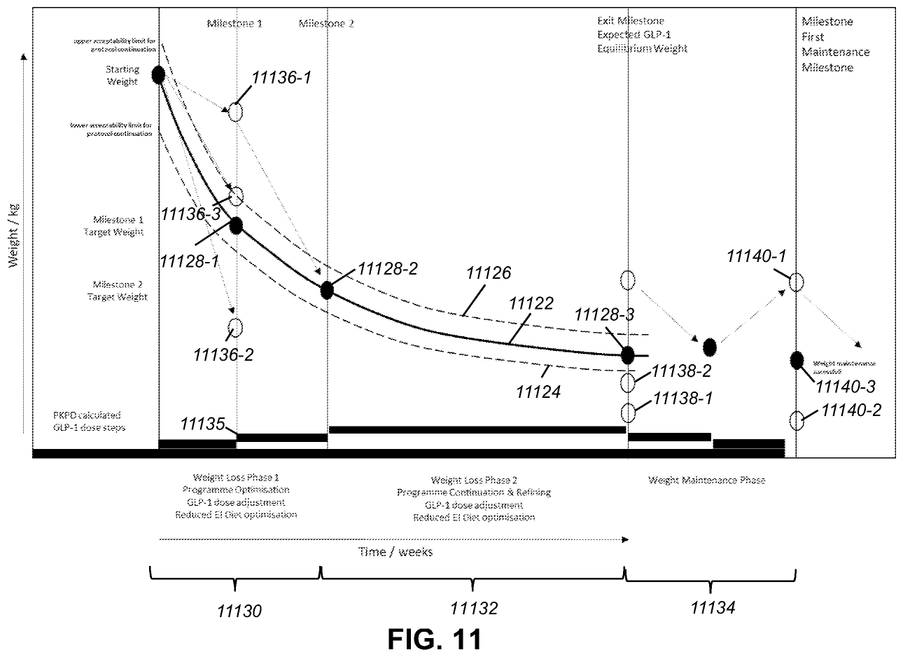

FIG. 11 illustrates the phased weight loss co-therapy in relation to an expected weight loss trajectory 11122 according to an embodiment of the present disclosure. The weight loss expectation range (or acceptable weight loss trajectory) including the upper weight loss trajectory 11124 and the lower weight loss trajectory 11126 is shown surrounding the weight loss trajectory 11122. A series of weight loss milestones 11128-1, 11128-2, 11128-3 can be seen on the expected weight loss trajectory 11122.

The expected weight loss trajectory 11122 is plotted against time and divided into the initial treatment titration phase 11130, the progressive weight loss phase 11132 and the treatment maintenance phase 11134. A GLP1 dosage (regimen) 11135 is shown as a stepped introduction and withdrawal across the three phases.

Progress data indicating different patient response trajectories can be seen at some of the weight loss milestones to exemplify the method response. For example, at a time corresponding to the first weight loss milestone 11128-1, the method may receive first progress data 11136-1 for a first patient. The first progress data 11136-1 illustrates a weight loss that is less than the lower weight loss trajectory 11126. This may be indicative of: poor patient concordance with the GLP1 dosage regimen (e.g. due to side effects) or the calorie intake regimen (the exact cause may be identified by specific progress data (calorie intake data etc). The method may respond to the underperformance in weight loss by: increasing the GLP1 dosage (e.g. step 654 of FIG. 6 or 772 of FIG. 7); reducing the GLP1 dosage to reduce side effects (e.g. step 648 of FIG. 6); address poor calorie intake concordance (e.g. by providing an education model, recommending alternative diet plans, or seeking HCP input and potentially aborting treatment). The method can respond to improve the patient weight loss back within the acceptable weight loss trajectory.

As a second example, at a time corresponding to the first weight loss milestone 11128-1, the method may receive first progress data 11136-2 for a second patient. The first progress data 11136-2 illustrates a weight loss that exceeds the upper weight loss trajectory 11124. The method may assess whether the excessive weight loss is a risk to health (for example if the excessive weight loss may affect diabetes treatment). The method may respond by: maintaining the co-therapy regimen and continuing; reducing or eliminating the GLP1 dosage (e.g. step 662 or 768); reducing the calorie intake allowance further until at target; or (transitioning early to the maintenance phase 11134.

A third example at a time corresponding to the first weight loss milestone 11128-1, the method may receive first progress data 11136-3 for a third patient. The first progress data 11136-3 indicates weight loss is on track within the acceptable weight loss trajectory and the co-therapy regimen is maintained.

Patient progress data 11138-1, 11138-2 received at a time corresponding to the third milestone 11128-3 that indicates weight loss exceeding the lower weight loss trajectory indicates that target weight loss has been achieved within a target time (e.g. 44-60 weeks) and the patient can transition to the weight maintenance phase 11134.

As a further example, the method receives further patient progress data 11140-1 for a first patient at the end of the weight maintenance phase 11134 and after drug withdrawal that indicates patient relapse and weight gain above the target weight. The method may respond by re-entering GLP1 therapy or providing an episodic pulse of GLP1 treatment. The method may also reduce the calorie intake allowance to promote weight loss back to target weight.

Further patient progress data 11140-2, 11140-3 for a second patient and a third patient indicates successful completion of the treatment program.

Patient Examples

Two patient examples are described with continuing reference to the protocol method of FIGS. 6 to 9.

An example case is Mary, a 46 year old Caucasian of height 160 cm and weight 130 kg, leading to a BMI of 50.8 kgm2. Her target weight is 70 kg. Whilst she would still be overweight with a BMI of 27.3, this would be transformative. The current estimated calorie intake is 2600. A target calorie reduction of 900 calories less than baseline per day is calculated in order to reach the target weight. She commences the treatment therapy and GLP1 administration and during the first two weeks has high levels of hunger and continues to snack between meals, failing to achieve the required calorie restriction. Her motivation score is low. The method lowers the calorie restriction to 100 calories drop from baseline (step 654) while the GLP1 dose is titrated upwards according to the initial GLP1 dosage regimen. As the GLP1 takes effect, satiety levels become consistently high on moving average, at which stage the method adjusts the calorie restriction to a more stringent calorie level of 500 calories less than baseline (Step 6622). Satiety levels are maintained as GLP1 levels continue to increase according to the initial GLP1 dosage regimen and a full 900 calorie restriction introduced (step 662). The patient enters the progressive weight loss therapy with the maximum GLP1 dosage and the 900 calorie restriction. The weight gradually drops to 73 kg with no variation in GLP1 dosage or calorie restriction. As the weight loss plateaus out, the GLP1 dosage is titrated downwards (step 988) until satiety levels begin to slip (step 970). The GLP1 dosage is then increased to the most recent controlling dosage amount (step 972). This is maintained for a further three months before complete withdrawal. However, after six months, a major life event leads to food binging and previous bad habits returning. Step 973 triggers a reintroduction of GLP1 which is titrated up to a maintenance dose and continued for a further three months before withdrawing once more. At follow up one year later, weight remains steady.

Harold is a 50 year old male of height 1.86 m and weight 149 kg, giving him a BMI of 43.1. His desired weight is 86 kg, giving a BMI of 24.9, just within the normal range. He does not have access to any intense calorie restriction techniques such as food substitution and instead is initiated on a 200 calorie per day restriction based on assessment of low motivation. However, satiety is high on the second scheduled step-up of the GLP1 dosage and more intense calorie restriction is then introduced at 400 calories per day deficit (step 662). A single further step up in GLP1 dose maintains the level of satiety. As his weight reaches 110 kg, his energy levels and body pains have altered, such that he is able to introduce an exercise (physical activity) regimen (step 759) which is gradually titrated up in proportion to the continuing decrease in his pains as a result of his weight loss and his increasing energy levels. The patient continues in the progressive weight loss phase with no change in GLP1 dosage or calorie restriction and weight continues to drop until a plateau of 94 kg. A further dose titration is then introduced to allow a further calorie restriction by an extra 50 calories and then three months later a target weight of 86 kg is reached (combination of steps 776, 780, 770 and 772). The GLP1 is then withdrawn, but his calorie restriction slips to 200 calories as a result of lower levels of satiety. The GLP1 is reintroduced (step 972) at the starting dose of 0.25, which allows re-establishment of the greater calorie restriction. Funding ultimately means this cannot be maintained and he ultimately has a long term calorie restriction delivering a weight of 94 kg.

Although the above examples is described in relation to a co-therapy comprising a single incretin pathway drug and a behavioural regimen, it will be appreciated that other examples may include more than one incretin pathway drug and/or one or more other obesity treatment drugs. For example, an the co-therapy may comprise an additional obesity treatment drug such as metformin or topiramate. In some examples, the method may comprise setting, determining and/or adjusting the dosages of a plurality of obesity treatment drugs in response to the patient data.

FIGS. 5 to 11 and their associated description are merely exemplary and do not limit the scope of the invention which is defined by the appended claims. One or more steps may be optional. Furthermore, the instructions and/or flowchart steps can be executed in any order. Also, those skilled in the art will recognize that while one example set of instructions/method has been discussed, the material in this specification can be combined in a variety of ways to yield other examples as well, and are to be understood within a context provided by this detailed description.

In some example embodiments the set of instructions/method steps described above are implemented as functional and software instructions embodied as a set of executable instructions which are effected on a computer or machine which is programmed with and controlled by said executable instructions. Such instructions are loaded for execution on a processor (such as one or more CPUs). The term processor includes microprocessors, microcontrollers, processor modules or subsystems (including one or more microprocessors or microcontrollers), or other control or computing devices. A processor can refer to a single component or to plural components.

It will be appreciated that any reference to “close to”, “before”, “shortly before”, “after” “shortly after”, “higher than”, or “lower than”, etc, can refer to the parameter in question being less than or greater than a threshold value, or between two threshold values, depending upon the context.

It will be appreciated that any of the methods described herein, or parts thereof, can be encoded by computer-readable instructions and stored on a non-transitory computer-readable medium. Any part of the invention as described above can thus be implemented by a computer executing appropriate instructions stored on a non-transitory computer-readable medium. A computer readable medium storing such instruction is thus also within the scope of the present invention.

The foregoing discussion discloses embodiments in accordance with the present invention. As will be understood, the approaches, methods, techniques, materials, devices, and so forth disclosed herein may be embodied in additional embodiments as understood by those of skill in the art, it is the intention of this application to encompass and include such variation. Accordingly, this disclosure is illustrative and should not be taken as limiting the scope of the following claims.

1. A method for treating obesity in a patient, the method comprising:
administering a dose of a GLP1 agonist to the patient in a treatment regimen; and,
in conjunction with the administration of the dose of GLP1 agonist, engaging in a directed digital therapeutic program that manages patient weight loss and the treatment regimen for the GLP1 agonist with the objective of achieving a predetermined target weight for the patient.

2. The method of claim 1, wherein the digital therapeutic program comprises a behavioural regimen for managing the patient weight loss.

3. The method of claim 2, wherein the digital therapeutic program synchronises a timing of the behavioural regimen with a timing of the administration of the dose of the GLP1 agonist.

4. The method of claim 2, wherein the behavioural regimen comprises a calorie intake regimen and/or a physical activity regimen.

5. The method of claim 1, wherein the digital therapeutic program manages the patient weight loss and the treatment regimen by:
receiving a patient initial weight; and
setting a calorie intake regimen based on the patient initial weight and the target weight.

6. The method of claim 1, wherein the digital therapeutic program manages the patient weight loss and the treatment regimen by:
setting a calorie intake allowance of the calorie intake regimen based on a GLP1 dosage of the treatment regimen.

7. The method of claim 6, wherein the method sets a timing of the calorie intake regimen in accordance with a timing of a satiety effect of the GLP1 dosage.

8. The method of claim 1, wherein the digital therapeutic program manages the patient weight loss and the treatment regimen by:
receiving patient progress data comprising one or more of: patient weight loss data, patient calorie intake data, patient motivation score, patient satiety score, patient side effect data and patient activity data;
adjusting a GLP1 dosage of the treatment regimen and/or the calorie intake regimen based on the patient progress data.

9. The method of claim 8, comprising adjusting the GLP1 dosage if an elapsed time exceeds a dosage effect time threshold.

10. The method of claim 8, wherein the digital therapeutic program manages the patient weight loss and the treatment regimen by:
during a weight loss phase of the treatment, adjusting a GLP1 dosage of the treatment regimen and/or the calorie intake regimen based on the patient progress data to provide a weight loss trajectory towards the target weight.

11. The method of claim 8, wherein the digital therapeutic program manages the patient weight loss and the treatment regimen by transitioning from a weight loss phase to a weight maintenance phase if the patient weight loss data indicates a rate of weight loss has been less than a threshold weight loss rate for a period of time exceeding a stability time threshold.

12. The method of claim 8, wherein the digital therapeutic program manages the patient weight loss and the treatment regimen by:
during a weight maintenance phase of the treatment, adjusting a GLP1 dosage of the treatment regimen and/or the calorie intake regimen based on the patient progress data to maintain the patient weight within a threshold range of the target weight.

13. The method of claim 8, wherein the digital therapeutic program manages the patient weight loss and the treatment regimen by:
during a weight loss phase of the treatment or a weight maintenance phase of the treatment, adjusting a GLP1 dosage of the treatment regimen to maintain the patient satiety within a threshold patient satiety range.

14. The method of claim 8, wherein the digital therapeutic program manages the patient weight loss and the treatment regimen by:
during a drug withdrawal phase of the treatment, reducing a GLP1 dosage of the treatment regimen and adjusting the calorie intake regimen based on the patient progress data to maintain the patient weight within a threshold range of the target weight and stop administration of the GLP1 agonist.

15. The method of claim 8, wherein the digital therapeutic program manages the patient weight loss and the treatment regimen by:
increasing a calorie intake allowance of the calorie intake regimen if:
the patient calorie intake data represents a calorie intake greater than a first upper calorie intake threshold;
the patient motivation score is less than a motivation score threshold; or
the patient satiety/hunger score is less than a first lower patient satiety threshold.

16. The method of claim 8, wherein the digital therapeutic program manages the patient weight loss and the treatment regimen by:
increasing a calorie intake allowance of the calorie intake regimen and increasing a GLP1 dosage of the treatment regimen if:
the patient calorie intake data represents a calorie intake greater than a second upper calorie intake threshold;
the patient motivation score is less than a motivation score threshold; or
the patient satiety score is less than a second lower patient satiety threshold.

17. The method of claim 8, wherein the digital therapeutic program manages the patient weight loss and the treatment regimen by:
increasing a GLP1 dosage of the treatment regimen if:
the patient weight loss data represents a weight loss that is less than an acceptable weight loss trajectory;
the patient calorie intake data represents a calorie intake greater than a third upper calorie intake threshold; or
the patient satiety score is less than a third lower patient satiety threshold.

18. The method of claim 8, wherein the digital therapeutic program manages the patient weight loss and the treatment regimen by:
decreasing a GLP1 dosage of the treatment regimen if:
the patient weight loss data represents a weight loss greater than an acceptable weight loss trajectory; or
the patient side effect data is representative of a level of side effects greater than a side effect intolerance threshold.

19. A method for providing a co-therapy to a patient suffering from obesity, wherein the co-therapy comprises a GLP1 agonist for administering to the patient according to a treatment regimen and a digital therapeutic program comprising a behavioural regimen for administering using an electronic device, wherein the method comprises:
receiving personalised patient data;
setting a GLP1 dosage of the treatment regimen based on the personalised patient data;
setting a calorie intake regimen of the behavioural regimen based on the personalised patient data;
outputting the GLP1 dosage and the calorie intake regimen.

20. An apparatus comprising one or more processors and computer readable memory including instructions which when executed by the one or more processors carry out the method of claim 19.
